(19) United States
(12) Patent Application Publication
(10) Pub. No.: US 2015/0191370 A1
(43) Pub. Date: Jul. 9, 2015

(54) DEVICE FOR TREATING A LIQUID

(71) Applicant: JMY INVEST APS, Copenhagen Ø (DK)

(72) Inventors: Tor Reedzt, Vejle (DK); Simon Joachim Birch Broni, Vejle (DK)

(73) Assignee: JMY INVEST APS, Copenhagen ø (DK)

(21) Appl. No.: 14/404,555

(22) PCT Filed: May 29, 2013
(86) PCT No.: PCT/EP2013/061138

§ 371 Date: Nov. 28, 2014

Related U.S. Application Data
(60) Provisional application No. 61/652,420, filed on May 29, 2012.

(30) Foreign Application Priority Data
May 29, 2012 (DK) PA 2012 70283

(51) Int. Cl. C02F 1/467; A61K 33/00; C02F 1/461

(52) U.S. Cl. CPC C02F 1/467; C02F 1/46109; A61K 33/00; C02F 2001/46171; C02F 2201/48; C02F 2303/04; C02F 2303/22; C02F 2209/40; C02F 2209/02; C02F 2209/36; C02F 2209/30; C02F 2209/055

(57) ABSTRACT

The invention provides an instant type liquid treating device which comprises a housing with an inlet and an outlet connected by a passage for passing a liquid medium through the device. The device further comprises at least one set of cooperating separate conductive faces connectable to an AC-power supply and directly exposed to each other in the passage such that the liquid medium can be treated by way of its own electrical resistance between the faces. A control is provided to facilitate relative movement of the faces.

### FIELD OF THE INVENTION

The present invention relates to a device for treating a liquid, the device being an instant type treating device. More specifically, the invention relates to an instant type liquid treating device comprising a housing with an inlet and an outlet connected by a passage for passing a liquid medium through the device. The device according to this invention comprises at least one set of conductive faces formed by electrodes, an outer one of the electrodes forming a channel in which the other, inner, electrode is received.

To provide an electrical field between the faces, the electrodes are connectable to an AC-power supply and the faces are directly exposed to each other in the passage such that the liquid medium can be treated by way of its own electrical resistance between the faces. The device further comprises a control facilitating movement of the faces relative to each other.

### BACKGROUND OF THE INVENTION

Traditionally, instant type devices are used to treat a liquid by passage of electrical current between two electrodes immersed in liquid, such as water, thereby utilising the conductivity of the liquid as a resistance element, when applying an electrical potential between the two electrodes.

As the conductivity of a liquid may depend on the site at which the liquid is found or on the liquid itself, the efficiency of the device may vary and control can be difficult. At some sites or with some liquids, a traditional instant type device may not even work due to the conductivity of the liquid at this specific site.

Therefore, a traditional instant type device is best usable in closed circuits where the conductivity of the liquid is known, or where the conductivity can be regulated. Thus, this traditional type cannot be used in connection with liquid supply, e.g. such as tap water, which often have a varying conductivity.

Generally, the traditional type devices are controlled by controlling the electrical charge of the electrodes. This way of controlling the device is complicated, costly, and often not very precise, and as a result, it is typically not possible to obtain a specifically desired effect of the treatment of the liquid medium, e.g. to obtain a specific temperature etc.

### DESCRIPTION OF THE INVENTION

It is an object of embodiments of the invention to provide an improved device for treating a liquid, an improved method of treating a liquid, and a liquid medium which is treated by the device and/or according to the method. Further, it is an object to provide a number of new fields of application for direct liquid heaters.

According to a first aspect, the invention provides an instant type liquid treating device where at least one of the outer and the inner electrode comprises at least two individual elements axially movable relative to each other via a control which thereby facilitate relative movement of the faces.

Due to the ability to move one element relative to the other element, adjustment of the energy which is transferred to the liquid medium can be varied in a very precise and simple manner. As a result, the device may be controlled based on a desired effect of the treatment, e.g. controlled to obtain a specific temperature of the liquid medium or to obtain other specific effects of the treatment. This opens up a new way of treating liquid media by electrical current through the media, and particularly, opens for a plurality of new fields of application for this technology.

The device may be controlled entirely based on the claimed movability of at least one element of the electrode relative to the other element s of the electrode. In one embodiment, the device may therefore have the movability of the elements relative to each other as the only possible means for control. In this embodiment, the device is supplied with that AC power which is available and the desired effect is controlled via the control by movement of the elements relative to each other.

The device comprises a control which facilitates relative movement of the elements, thereby enabling changing of the electrical potential between the at least one set of cooperating separate conductive faces, control of flow, maximum power use, whereby the treatment of the liquid may be adapted to a specific environment and thus type of liquid, e.g. quality of the liquid.

Thus, the relative movement of the faces can be used to counteract a changed output of the device as a result of e.g. a changed conductivity of a liquid, movement of the unit from one site to another site, as the relative movement may be adjusted in response to e.g. the conductivity, temperature, the microbial count, or other relevant measures, e.g. hardness of the liquid, mineral content, e.g. based on Calcium, or based on a measure significant for quality of the water, e.g. significant for taste or health, etc.

A liquid medium, such as water to be treated, flows into the housing via the inlet and passes through the passage to the outlet via which the liquid flows out of the housing again. I.e. the passage forms a flow-path for the liquid medium.

The liquid medium is treated by way of its own electrical resistance, as the device utilises the conductivity of the liquid medium as a resistance element, thereby treating the liquid medium by applying an electrical potential between the at least one set of cooperating separate conductive faces when connecting them to an AC power supply.

It should be understood, that by treatment of the liquid medium is in this connection meant heating of the liquid and/or a changed composition of particles, minerals, salt, microbes, and other objects which may be present in the liquid to be treated. The latter is herein referred to as quality improvement of the liquid, since it typically corresponds to cleaning or disinfection or sterilisation of the liquid. In relation to changing the composition of particles, enrichment, enhancement of taste, reducing of half-life, killing of bacteria, or destruction of vira, the liquid medium is not necessarily heated significantly.

If no heating is desired, it is within the scope of this invention also to combine the device with a cooling structure adapted to cool down the liquid medium such that the liquid medium has the same temperature at the inlet and at the outlet, or such that it is even colder at the outlet. The device may e.g. be combined with a compressor based cooling system.

The device may be used for various purposes for treatment of nearly any kind of liquid medium. The treatment, particularly when controlled, may at least provide the following effects on the liquid medium: heating, reduction of microbial count, reduction of a limescale developing effect, increase of hydrogen, and change of taste.

By reduction of a limescale developing effect is herein meant that the liquid medium becomes less limescale developing, e.g. when used in thermal devices where the liquid medium is heated, e.g. when used in a coffee maker, or washing machine etc. Without being bound by theory, it is believed that the reduction of the limescale developing effect is caused by a reduction of a content of limescale developing elements in the liquid substance. It may e.g. be caused by decomposition of limescale developing ions etc. Accordingly, the reduction of a limescale developing effect can be achieved by decomposition of, or by reducing the content of a limescale developing ion in the liquid medium.

The liquid medium could e.g. be water such as regular tap-water or salt water, process water e.g. for cleaning, washing, watering, bathing, or soaking purpose, it. could be bio-liquids such as body fluid, e.g. blood, and it could be different kinds of beverages including beer, soda water, alcoholic beverages such as wine, fruit juice and milk products.

The device can have a relatively low weight and low power consumption relative to its capacity for treating the liquid medium. Accordingly, it becomes applicable in many different places, e.g. in domestic properties, in hospitals, where sterility is an issue, and in mobile applications such as in boats where power consumption is an issue, and in places where space, ease of maintenance and reliability is an issue, e.g. in military installations, or in aircraft and space industry.

Due to the control by relative movement of the elements, the device may operate and different AC-signals, i.e. by use of the control, the elements can be moved, and the device may therefore provide the desired effect with different AC power supplies and with different AC signals on the electrodes. Accordingly, the device is suitable e.g. in combination with renewable energy. As an example, the device may be powered by solar energy, wind energy or other renewable energy sources, and it may be operated at different voltages depending on the availability of the renewable energy source.

The device is not limited to a specific size of fuse or circuit breaker due to the scalability of the device, simply the outcome depends on the availability of electrical power.

The device may comprise powering means for and generally be adapted for connection to a power source of standard type, e.g. 230 V-50 Hz or 110V-60 Hz, or other combinations within traditional grid supply. Particularly, the conductive faces may be supplied directly from such a grid, i.e. with a voltage in the range of 100-400 V, and with a relatively large current, e.g. in the range of 0.1-100 Amperes or more. The conductive faces may even be supplied with 12 or 24 V, e.g. for battery powered operation.

Additionally, the device may comprise a converter or a transformer, such that any available type of power can be utilised e.g. a battery of the kind used in cars, boats, and campers.

By controlling the relative movement of the conductive faces, the same device may be used both in connection with a power source of e.g. 230 V-50 Hz and in connection with a power source of e.g. 110V-60 Hz, thus providing an adaptive control that can be used throughout the world.

The device may comprise a filter adapted to retain particles, dead microbes and other dead organisms, and other objects being present in the liquid. The filter could be replaceable to facilitate different needs and thus different filter characteristics.

Two or more devices may possibly be connected in series in order to ensure efficient treatment, e.g. for killing bacteria. Particularly, it may be an advantage to connect two or three devices serially, and to connect them to different phases, e.g. to three different phases of a 3-phase AC power supply.

To further prevent bacteria formation or generation of a bio-film internally in the passage, the device may comprise means for entering treated liquid and/or a disinfecting or a detergent solution into the passage. In one embodiment, the device comprises a return loop from the outlet to the inlet to return purified liquid for removal of bio-film. The device may further be used to prevent or remove bio-film in a tube downstream of the device, as treated liquid from the device may remove the bio-film from the tube.

Consequently, the device may be used in example households, office buildings, and hospitals, public buildings where it is desired to kill e.g. e-coli, legionella or other bacteria in the water. Alternatively, the device may be used in process industry, developing countries or in disaster areas where the existing water supply is contaminated. As an example, the device may be coupled to a water pump and a power generator, such as a diesel generator, solar panel or windmill and then pumping water through the device, thereby disinfecting the water.

The power which is converted in the liquid depends on the voltage supplied to the faces, and the current between the faces, the conductivity of the liquid, the surface area of the least one set of cooperating separate conductive faces, the distance there between, flow and the used materials

As an example, the device may be subjected to a liquid with conductivity in the range of 25-80000 micro Siemens Amperes per Volt.

Depending on the voltage supply, the conductive faces can be connected to the power supply in various ways. If the voltage supply is a single-phase power supply, the phase will be connected to one of the conductive faces and the neutral conductor to the other one of the conductive faces.

By a two-phase power supply, the conductive faces will be connected to each of the phases in the power supply such that a potential difference arises between the two faces.

By a three-phase power supply there may e.g. be formed a so-called delta connection or a star connection. In a delta connection, the device includes three sets of conductive faces and one conductive face from each of the sets is connected to each their phase. The conductive faces are therefore grouped in three groups. The phases are coupled to the conductive faces such that a potential difference occurs. In a star connection, the one conductive face of each of the sets are interconnected and not connected with the power supply. The other one of the conductive faces from each of the sets are then connected to the three phases such that a potential difference occurs across the first ones of the conductive faces as the latter function as a neutral point. The device can be changed from one connection type to another with a switch e.g. no need of new wiring of the device.

The device may further comprise an amp meter or watt meter for measuring electric power consumption. The power converted in the electrically conducting liquid and thereby the temperature is almost proportional with the current running through the liquid. By including an amp meter, the power consumption of the device can be measured as the supplied voltage can be known and may be constant.

Knowing the converted power may be particularly important when the device is used in connection with treating of water from a water supply as the conductivity of the water may vary. The relative movement of the conductive faces may thus be carried out until a desired power and thereby temperature is attained.

In one embodiment, the control may facilitate both a change in an amount of confronting areas of the faces and spacing between the confronting faces thereby allowing for a more fine-grade control as both or only one of the area and the spacing may be changed dependent on the actual conditions.

By confronting areas is meant areas of the conductive faces which are directly opposite each other, so that the two conductive faces in a cross-section transverse to the flow-path form an overlap. It should be understood, that the control facilitating relative movement of the conductive faces, in one embodiment may be able to move the faces relative to each so that there in at least one position of the faces in no overlap, i.e. the confronting area is zero in this position.

The conductive faces may in one embodiment be parallel to each other and thus parallel to a flow-path formed between the conductive faces. In the following, reference is made to the part of the flow-path being positioned between the conductive faces as the effective flow-path.

In order to facilitate a simultaneous change in an amount of confronting areas of the faces and the spacing between the confronting faces, one of the confronting faces may comprise at least a surface portion which is non-parallel to at least a portion of the other face. When the conductive faces are shifted relative to each other along the effective flow-path, the distance between the conductive faces will vary along the effective flow-path due to the oblige angle.

The faces may be arranged so that they are shifted in linear relationship, e.g. by use of a linear motor, a spindle motor, by use of a thermo-sensitive linear element which provides linear displacement as function of temperature or by any other means capable of shifting the faces linearly relative to each other.

One way of achieving shifting of the faces in a linear relationship, is to provide the faces so that they are formed by electrodes which are cylindrical and relatively shiftable in axial direction. The cross-sectional shape of the faces may however in one embodiment be non-circular, such as oval.

Cylindrical electrodes may be provided such that an outer one of the electrodes forms a channel in which the other, inner, electrode is received. Thus, one of the electrodes may be arranged as a cylinder around the other electrode which may be formed as a cylinder or a solid core. It should be understood, that the channel and/or the inner electrode may have a cross-sectional shape being circular, oval, square-shaped or any of any arbitrary shape, arranged in a telescope like formation

The electrode forming the outer channel may be continuously or non-continuously formed. By non-continuously formed should be understood, that the electrode comprises e.g. openings, or through holes, such as perforations of a predetermined size. The size of the openings or through holes may vary along the length of the electrode or may be of a substantially uniform size. Furthermore, the openings or through holes may be uniformly or non-uniformly distributed over the electrode.

One way of achieving a surface portion of one conductive face which is non-parallel to a portion of the other conductive face, could be to provide the outer electrode so that it comprises a conical section. When the conductive faces are shifted relative to each other along the effective flow-path, the distance between the conductive faces will vary along the effective flow-path due to the conical section of the electrode forming the outer channel.

Alternatively, the inner electrode may be formed so that is comprises a conical section, as this may also result in a varying distance between the conductive faces when shifting them relative to each other along the effective flow-path.

In one embodiment, the conical section widens out in a direction in the passage towards the inlet. However, in an alternative embodiment, the conical section widens out in a direction towards the outlet, as the conductivity of the liquid may increase when increasing the temperature of the liquid. Due to the increased conductivity, the distance between the two electrodes can be increased, e.g. by applying a conical section of the outer electrode which widens out in a direction towards the outlet.

The outer electrode may be arranged so that it can be shifted axially in the passage, thereby allowing for relative movement of the conductive faces. In one embodiment, the inner electrode may also be shiftably arranged. However, in an alternative embodiment, only the inner electrode may be shiftably arranged.

Thus, the relative movement of the faces may at least partly be provided by relative movement of the at least two individual cylindrical elements constituting the outer or inner electrode.

The control may comprise either an electrically operated motor or otherwise power driven means for moving the individual elements axially relative to each other. The motor may e.g. be a step motor. For providing a simpler device, the control may, however, also comprise a manually operated handle, e.g. in the form of a rotatable knob, e.g. a knob which rotates a threaded element which in response causes axial translation of the individual elements relative to each other.

At least one of the outer and inner electrodes may comprise at least two individual faces which are electrically isolated from each other and connectable to different phases of the AC-power supply. As an example, the outer electrode, or the inner electrode may comprise three different faces which are connected to different phases of a three-phase power supply. If the electrode in question is tubular or otherwise has a circular cross-section, the three phases may form in the range of up to nearly 120 degrees peripherally along the circular periphery.

The inner electrode or the outer electrode comprises at least two individual cylindrical elements axially movable relative to each other. Thus, the at least two individual cylindrical elements may by arranged in a telescope-like arrangement, thereby enabling relative movement of the outer electrode and the inner electrode relative to each other by changing the effective length of that one of the outer and inner electrode which is telescope-like. In the following, we refer to “the telescope-like electrode” as being that one, or both of the inner and outer electrodes which are constituted by elements in a telescope-like arrangement.

By telescopic layout is herein meant that the at least two individual elements are located one within the other thereby forming an inner element and an outer element in such a manner that the inner element can slide axially relative to the outer element. The elements may e.g. be tubular elements e.g. with a circular cross-section.

By effective length of the outer electrode should be understood the actual length of the telescope-like electrode in a given position of the least two individual cylindrical elements relative to each other. Furthermore, the distance between the inner and outer electrode can be changed, due to the different diameter of the individual cylindrical element. In one embodiment both the inner electrode and the outer electrode comprises individual elements, e.g. in a telescope-like arrangement.

In the telescopic layout, one element becomes the outer and one becomes the inner element, and a number of additional elements may be arranged one within the other between the outer and the inner element, herein we refer to these elements as “intermediate elements”.

The telescope-like electrode is movable between a collapsed state and an expanded state by movement of the elements relative to each other. When the telescope-like electrode is in the collapsed state, the elements are all located one within the other, and the effective length of the telescope-like electrode is reduced. When the telescope-like electrode is in the expanded state, the elements are axially off-set relative to each other, and the effective length of the telescope-like electrode increases. Simultaneously, the overall shape of the telescope like electrode becomes conical.

In combination with a telescope-like electrode, the other one of the inner and outer electrode may also be conical. In this embodiment, the inner and outer electrodes may preferably be oriented in the same way with the narrow end of the conical shape pointing in the same direction.

In one embodiment, the inner electrode may have a conically shaped section, as mentioned above. Such an inner electrode may be combined both with an outer electrode comprising a conical section and with an outer electrode comprising at least two individual cylindrical elements axially movable relative to each other.

The conically shaped section of the outer or inner electrode may narrow down in a direction towards the inlet. However, in an alternative embodiment, the conical section widens out in a direction towards the inlet, as the conductivity of the liquid may increase when increasing the temperature of the liquid. Due to the increased conductivity, the distance between the two electrodes can be increased, e.g. by applying a conical section of the inner electrode which widens out in a direction towards the inlet.

In one embodiment, the outer or inner electrode may have a conically shaped section, as mentioned above. Such an outer electrode may be combined both with an inner electrode comprising a conical section and with an inner electrode comprising at least two individual cylindrical elements axially movable relative to each other.

The conically shaped section of the outer electrode may narrow down in a direction towards the inlet. However, in an alternative embodiment, the conical section widens out in a direction towards the inlet, as the conductivity of the liquid may increase when increasing the temperature of the liquid. Due to the increased conductivity, the distance between the two electrodes can be increased, e.g. by applying a conical section of the outer electrode which widens out in a direction towards the inlet.

To facilitate liquid flow in the passage, the inner or outer electrode may have a smooth surface. A smooth surface may further facilitate that particles, contamination, etc. have difficulties adhering to surface, thereby minimising the risk of lowering the efficiency of the device due to unwanted deposition hereof. Furthermore, the device does not need a larger surface area to ensure sufficient transfer of heat from a heating element as in traditional heaters in which heat is transferred by thermal convection, as this device utilises the conductivity of the liquid.

To further increase the performance of the device the outer or inner electrode may comprise of several individual elements in an arrangement with at least one electrode being telescope like in format, thus enabling a booster mode for even better performance where energy is available or where the conductivity of the liquid is low.

To increase safety, at least one of the inlet and outlet may be connectable to zero of a power supply or earth or in an addition following the path with earth connection a zero to a power supply connection, as this may prevent current leakage and thereby prevent that a person being in contact with the liquid which has been treated is getting an electric shock.

To increase safety further, the device may additionally comprise at least one extension-tube of a non-conductive material extending from at least one of the inlet and the outlet, the extension tube terminating in a coupler of a conductive material. The coupler may be electrically connectable to a consumer of electricity or to ground of a power supply, thereby preventing a person getting an electric shock in case of current leakage, e.g. a HPFI, Residual-Current-Circuit-Breaker, or an earth leakage circuit breaker. Even further a zero of a power supply may be connectable after the path with earth connection, thus preventing leakage.

The electrical connectivity of the coupler to the ground connector or circuit breaker may in one embodiment be established by a wire extending inside the extension-tube.

At least one of the electrodes may be made from Stainless steel, e.g. A4, may further be selected, due to its corrosion resistance and antibacterial properties.

Alternatively, at least one of the electrodes may be made from copper. Copper has good electrical conducting properties, and it is cheap while at the same time allowable for use in consumer systems coupled to a common water supply, such as tap water.

However, other kinds of electrically conducting material, such as black iron, silver, gold, carbon, graphene and alloys may also be used for the conductive faces or for the entire electrode s.

On the contrary, the housing may be made of a heat resistant material. The housing can be made of a moulded or extruded heat-resistant material, or a heat-resistant material worked in other ways, such as plastic, fibre, fibre-reinforced plastic and similar.

The device may further comprise an electronic circuit facilitating phase angle control or pulse generation. This facilitates regulation of the current, and thereby the power converted in the liquid.

By phase angle control is meant, that the phase angle between current and voltage is changed. By use of an AC power supply, the power converted will be changed, when changing the phase between current and voltage, thereby allowing for a further fine-grading of the control of the relative movement of the conductive faces.

By pulse generation control is meant, that in addition to the relative movement of the conductive faces, pulsing is used to achieve the output by control of the pulse repetition rate, the pulse width, a delay with respect to a trigger, and/or the high- and low-voltage levels of the pulses. Furthermore, the control may be based upon the rise time and/or fall time of the pulses.

The electronic circuit may further facilitate frequency control by use of AC-DC-AC conversion of the current. The frequency of the current between the at least two conductive faces may determine the amount of power converted in the liquid.

An alternative or cooperating, to relative movement of the conductive faces, control strategy may be switch mode control. By switch mode control is meant, that the voltage is regulated based on a measurement of power of current to arrive at the required current between the at least two conductive faces.

To facilitate control of the relative movement of the at least two conductive faces, the device may comprising a sensor for sensing a parameter significant for the liquid medium, and means for adjusting at least one of the power supply and the relative movement of the faces based on the sensed parameter. The parameter may e.g. conductivity, temperature, flow, and current.

The controller may e.g. be a proportional P, a proportional integral PI, a proportional differential PD, or proportional integral differential PID controller. In one embodiment, the controller may perform closed loop control. I.e. a chosen controlling parameter may be measured and/or sensed and subsequently fed back to the controller so that the relative movement of the faces can be based on the difference between the measured/sensed feedback value and the input value of the controlling parameter. It should be understood, that the chosen parameter used as feedback value need not be a single parameter, but may be a set of parameters used together.

A transfer function of the controller may be a function of one or more controlling parameters selected from a group consisting of: current, voltage, conductivity, temperature, flow, liquid hardness, and liquid quality. I.e. one or more controlling parameters may be measured and/or sensed and subsequently fed back to the controller so that the relative movement of the faces can be based on the difference between the measured/sensed feedback value of the controlling parameter s and the requested value of the controlling parameter s.

In order to ensure fully treatment of the liquid medium flowing between the inlet and the outlet, the faces may be located relative to the passage such that an entire amount of the liquid medium flowing between the inlet and the outlet passes between the faces. This may be especially important if the device is used for cleaning or disinfection/sterilisation purposes, as an amount of liquid being by-passed or not passing between the faces constitute a potential risk for the end-user of the liquid.

To be able to deliver the liquid instantly at a required temperature, the device may comprise a shunt loop facilitating return of at least a portion of liquid medium which exits the outlet back to the inlet. This may ensure that an amount of liquid which has no longer or has not yet arrived at the required temperature is not delivered to the end-user but is instead returned to the inlet for further treatment for instant delivery of the liquid at the correct temperature. Further to be able to deliver the liquid instantly the outlet may be arrange as close as possible to the treatment of the liquid, thus no liquid is left untreated as it would be if the treatment process takes place at the inlet.

The device may comprise a controlled valve, e.g. a servo valve, e.g. electrically operated, and located to control the flow of the liquid medium through the passage. The valve could e.g. be controlled based on a control parameter which is indicative of:
a desired flow of the liquid medium through the passage; or
a desired temperature of the liquid medium; or
a desired reduction of a microbial count in the liquid medium; or
a desired increase in content of hydrogen in the liquid medium; or
a desired reduction in the limescale developing effect of the liquid medium.

As the conductivity may vary heavy dependent on the liquid, the device may be formed so that at least one of the faces is releasably attached to facilitate replacement. Thereby, it may be possible to choose a conductive face which is applicable e.g. based on e measurement of the conductivity of the liquid in question.

To increase the amount of liquid being treated, the device may comprise several passages and corresponding sets of separate conductive faces, the passages being arranged in parallel between the inlet and outlet.

In one embodiment, the relative movement of the conductive faces may be effected via a rotating spindle. To avoid current leakage, the spindle may be electrically isolated from the faces and/or it may be made from a non-conductive material or at least include a section of a non-conductive material. It should however be understood, that other means may also be applied.

Since bacteria are living organisms, they can be killed by electric shock or by any related effect of an electrical current through the water, such as vibration. Herein, we generally describe the effect as that provided by an electrical current through the water. A liquid which contains bacteria, DNA, virus, etc., can therefore be treated by the present device.

In a second aspect, the invention provides a method for treating a liquid medium where the liquid medium is exposed to an electrical field by use of the device according to the previous description and any of claims 1-31. Particularly, the method may relate to treating liquid until a reduction of a microbial count can be detected, until the content of hydrogen in the liquid medium is increased, or until the limescale developing effect of the liquid medium is decrease relative to the limescale developing effect of the liquid medium before it is exposed to the electrical field. In one embodiment the treatment by use of the device is carried out until a combination between the three effects is experienced, e.g. until both the limescale developing effect and the microbial count is reduced.

Microbial count in this connection means a measure of the number of reproducible vira and/or bacteria in the liquid medium.

Particularly, the device may comprise one or more sensors capable of determining hydrogen content, or a microbial content, or temperature, or a limescale developing effect of the liquid medium. The method may include the step of operating the control and thereby moving the elements of the device based on a signal from one or more of such sensors.

Particularly, the method may be carried out on a body fluid such as blood, on a beverage or at least a content of a beverage, on process water for cleaning purpose or for watering of plants. The method may therefore form an integrated part of an application selected from the group consisting of: “cars, airplanes, boats, caravans, radiators, irons, water heaters, coffee makers, ice cube makers, water chillers, beer taps, orange juice taps, soda water taps, high pressure washers, dish washers, washing machines for laundry, window washers, car washers, air plane washers, sprinkling systems for cars, sprinkling systems for fire extinguishing, and garden sprinkling systems.

The amperage required for killing the bacteria will vary, depending on the type of bacteria in the liquid. Furthermore, the flow of the liquid through the device will determine for how long time the bacteria are subjected to electric shock. By experiments, it has been found that an alternating current, i.e. an AC electrical signal, on the electrodes have proven to provide destruction of bacteria and vira. The liquid medium may particularly be exposed to the electrical field, and particularly to an AC-signal, until a reduction of a microbial count can be detected. The device may particularly use several phases connected to separate sets of electrodes arranged serially in the liquid flow.

By experiments, it has been found that an electrical field through water or other liquid substances may generate hydrogen in the substance or water. Particularly it has been found that an AC electrical signal on the electrodes may produce high amounts of hydrogen. Hydrogen can have an advantageous effect in many aspects.

By way of examples, hydrogen enriched water may prevent metabolic syndrome. A disorder characterized by a constellation of symptoms including obesity, insulin resistance, high cholesterol and hypertension, metabolic syndrome is associated with an increased risk for cardiovascular disease and type 2 diabetes. Specialists conducted a study of 20 patients at risk for metabolic syndrome, instructing them to drink about two quarts of Hydrogen Enriched Water per day for eight weeks. Blood tests were done at the start, middle and end of the study period. Results: After eight weeks, participants showed, on average, a 39% increase in blood levels of antioxidant enzymes, 8% increase in blood levels of HDL “good” cholesterol and 13% decrease in total cholesterol—levels of improvement that significantly lowered their risk for metabolic syndrome.

It may also improve health for diabetes and pre-diabetes patients. A Japanese study involved 36 patients with either type 2 diabetes or impaired glucose tolerance a pre-diabetic condition in which blood glucose levels are higher than normal. Some patients drank about 30 ounces of Hydrogen Enriched Water daily for eight weeks . . . the rest drank the same amount of plain water. Results: Hydrogen Enriched Water consumption was associated with significant decreases in LDL “bad” cholesterol and urinary markers of oxidative stress as well as improved glucose metabolism . . . in two-thirds of pre-diabetes patients, oral glucose tolerance test results returned to normal. Among plain water drinkers, there were no significant changes.

It may also ease the negative side effects of radiation treatment for cancer. In a 2011 study, concerning 49 liver cancer patients undergoing radiation, a treatment that often increases fatigue and negatively affects quality of life. Participants who drank about two quarts of Hydrogen Enriched Water daily for six weeks showed lower blood levels of oxidative markers by-products of cell injury caused by free radicals and reported higher quality of life than participants who drank tap water. Hydrogen Enriched Water did not compromise the radiation's therapeutic antitumor effects.

In addition, animal studies show that consumption of Hydrogen Enriched Water may help reduce the risk for atherosclerosis, prevent stress-induced declines in learning and memory, slow the progression of Parkinson's disease, prevent or ease colitis, reduce allergic reactions, improve kidney function in kidney transplant patients, and lessen kidney toxicity and other side effects of the chemotherapy drug cisplatin.

Accordingly, the liquid medium may particularly be exposed to the electrical field until the content of hydrogen in the liquid medium is increased, e.g. to a level of 150-900 percent of the hydrogen level before the liquid is treated by the device.

It has also been found by experiments that water and other liquid substances treated by the device becomes less limescale developing in various devices, e.g. in household devices for coffee making etc. Accordingly, the liquid medium may be exposed to the electrical field until the limescale developing effect of the liquid medium is decrease relative to the limescale developing effect of the liquid medium before it is exposed to the electrical field.

The liquid medium may particularly be a body fluid in vitro, e.g. blood, a content of a beverage such as coffee, tea, beer, soda water, or pure water for drinking purpose, or process water for cleaning purpose. Due to the nature of the device, i.e. that the treatment is based on the electrical conductivity of the liquid medium, the treatment takes into account the viscosity of the body fluid e.g. the viscosity of blood, and the device is highly scalable within flow, temperature, power consumption, volts/amps, phases and size.

In one embodiment the device may be used in a mobile application where the advantage of a lightweight and low power consuming device which can heat and disinfect is desirable.

Such mobile applications may include boats, caravans, and airplanes etc. It may also include a mobile radiator as an electric unit using AC to heat water. The mobile radiator offers no dry heating—dry air and no risk for setting fire to materials covering the unit due to dry air. Further, the unit may function as an Add-On device for refitting existing water radiators e.g. for making them mobile. A mobile application may also include a mobile water heater for instant production of hot water, e.g. for use at construction sites, for cleaning boats, cars and other outdoor applications.

For movable or mobile devices, the advantage of device includes that it will not require a separate water tank to hot water, it uses the water from cold water tank, and it heats the water instantly in a continuously flow. The device is able to produce hot water with less than 1 kW depending on the size of the device, and with an efficiency of 100%, except for the loos to wiring and cables.

The device will clean the treated water by reducing the microbial count. As a result, the use may benefit from water without bacteria and vira, even if the system has not been used for a period of time, or in warm environments.

Furthermore the solution may have a relatively low weight since no separate water tank is needed. Even further, the device is highly scalable within flow, temperature, power consumption, volts/amps, phases and size. In addition, the water will be hydrogen enriched which may have a positive effect on the taste and healthiness.

In a third aspect, the invention therefore provides the use of a device as previously described and in accordance with any of claims 1-31 for a mobile application e.g. selected from a group consisting of: cars, airplanes, boats, caravans, radiators, and water heaters.

In one embodiment the device may be used for treating a beverage, e.g. coffee, tea, beer, soda water, any kind of alcoholic beverages, fruit juice, milk and other beverages. The device may e.g. form part of a coffee machine, a beer or soda water tap, a water cooler, or it may even form part of an ice cube maker etc.

The coffee machine will produce hydrogen enriched coffee and has a cleaning ability or is anti-bacterial even at low temperature. The coffee achieves a taste enhancement from hydrogen enrichment of the water and due to AC direct heating, instead of indirect heating from a heating element. Further, there will be no dry fire of heating element and no limescale which is especially challenging at high ramp up in temperature in every brew for a traditional heating element. Even further, the heating will be close to instant and close to 100% efficient—except for the loss to wiring and cables, and the device is highly scalable which makes it possible to brew at low power consumption, low volts/amps/watts and at low flow rate.

In a fourth aspect, the invention therefore provides the use of a device as previously described and in accordance with any of claims 1-31 for a beverage related applications, e.g. for processing beverages or for dispensing beverages, particularly beverages selected from a group consisting of: coffee, water, beer, fruit juice, wine and other alcoholic beverages, and soda water.

The device may e.g. be used as a beverage dispenser. It could also be for dispensing hot beverages. Currently such dispensers use a traditional heat element, with known problems like limescale, buildup of bacteria in the water, possibility of dry fire and a low efficiency e.g. Water are kept warm even when there is no use, simply because otherwise the waiting time would be too long for the water to warm up. Our proposed method or embodiment delivers a scalable solution that not only heats the water but at the same time cleans the water from bacteria with energy efficiency of 100%—except for the loss to wiring and cables since no standby energy is consumed and due to the instant availability of hot water. Further, there is no risk of dry fire. Further, the device hydrogen enriches the water and is highly scalable within flow, temperature, power consumption, volts/amps, phases and size.

In a fifth aspect, the invention provides the use of a device as previously described and in accordance with any of claims 1-31 for a cleaning related application selected from a group consisting of: high pressure washers, dish washers, washing machines for laundry, window washers, car washers, air plane washers, sprinkling systems for cars, sprinkling systems for fire extinguishing, and garden sprinkling systems.

In a dishwasher or in a laundry washing machine, taken as an example, the device may provide faster or instantly heating, 100% efficiency—except for the loss to wiring and cables, no limescale, which means no need for salt and no need for a salt compartment, lower grease buildup due to continues hydrogen treatment, anti-bacteria, temperature ratio from inlet temperature to boiling point, lowering bad odor due to hydrogen treatment and no dry fire of heat element. Further, the device is highly scalable within flow, temperature, power consumption, volts/amps, phases and size. The device will instantly heat and treat the water in a continuous flow. In addition, the water will be softened by breaking down the limestone which increases the use/function of soap and the washing effect.

In a sixth aspect, the invention provides the use of a device as previously described and in accordance with any of claims 1-31 for pasteurising a beverage, e.g. milk and juice. The device offers microbial reduction at low temperatures, ability to use renewable energy, instant and milder treatment, and may be less space consuming. In addition, the beverages have longer shelf life and preserve much of the taste. Further, the device may change the chemical compound of the liquid, hydrogen enriches the liquid, and eliminates challenging bacteria such as i.a. Lactus Basilus, fungus, etc. Even further, the device is highly scalable within flow, temperature, power consumption, volts/amps, phases and size.

In a seventh aspect, the invention provides the use of a device as previously described and in accordance with any of claims 1-31 for reducing half-life of a radioactive fluid substance. The device lowers the half-life by Hydrogen treatment. A continues treatment of the liquid medium will enhance the results and lower the half-life. For use at power plants the power is available from the power plant. The device is suitable for decontamination systems for military use. Further, the device is highly scalable within flow, temperature, power consumption, volts/amps, phases and size.

In a eights aspect, the invention provides the use of a device as previously described and in accordance with any of claims 1-31 for waste water treatment. Current methods use chemicals to lower the e-coli and coliform bacteria number by adding very toxic mixture to the water just before releasing it in to a sand filter that leads the water into the nature. Our improved method improves this process by letting the water flow through the device, where it is directly energized using AC current. The AC current could be generated by windmills, solar panels or other green energy concepts. This cleans the water from all bacteria including e-coli without the use of any chemicals, thus the environment is not damaged. Furthermore the e-coli level can be brought down to a lower level. If the waste water is contaminated e.g. with radioactive waste, the half-life will be reduced by the added hydrogen that binds the free radicals thus improving the quality of the waste water. Current methods use chemicals and a process where the radioactive water is mixed with a dry solution e.g. black concrete, this makes for easier storage but does not eliminate the problems with nuclear waste. Further, the device is highly scalable within flow, temperature, power consumption, volts/amps, phases and size.

In a ninth aspect, the invention provides the use of a device as previously described and in accordance with any of claims 1-31 for cleaning of teat-cups of a device for milking an animal or for cleaning the teats of the animal. Particular, the device may be used for providing a treated liquid medium for cleaning of sensitive areas, e.g. for cleaning teat cups, the udder of the animal and other sensitive areas thereby removing potential bacteria e.g. removing the potential for transmitting pathogenic microorganisms that might cause mastitis. This may prevent infection from one animal to another intra-mammary infection and the milk will be less impure. Currently hot water and chemicals are used for the same purpose. The solution according to this invention uses less energy as it is typically not necessary to provide boiling water to remove impurity. This results not only in cost reduction but also reduces the time it takes to clean. Further, the device hydrogen enriches the water and is highly scalable within flow, temperature, power consumption, volts/amps, phases and size.

In a tenth aspect, the invention provides the use of a device as previously described and in accordance with any of claims 1-31 for preparing a liquid medium for sprinkling of fruits and vegetables. The spraying onto the vegetables may insure that bacteria e.g. e-coli, coliform bacteria and legionella are removed from the water before being sprayed via an atomizer. Currently cold water is used, but without being used all the time bacteria can build up along with biofilm in the piping. In groceries, consumers are present below the atomizers when in use, which further enhances the problems should legionella or any other bacteria be present. The improved method does not rely on chemicals for cleaning the water and is able to enhance the temperature giving the consumers a more pleasant feeling when sprayed by the atomizers. Further, the device hydrogen enriches the water and is highly scalable within flow, temperature, power consumption, volts/amps, phases and size.

The device may also be used for watering plants, vegetables, fruits etc. The device offers increased growth without the use of chemicals and/or fertilizer. The device is not space consuming, it is easy to use and gives an instant production in a continuous flow of water or water containing liquid. The device hydrogen enriches the water. Further, the device is highly scalable within flow, temperature, power consumption, volts/amps, phases, and size. Even further, it is believed that the water molecules after treatment are amended such that the plants can better absorb the water and thus grow faster.

In an eleventh aspect, the invention provides the use of a device as previously described and according to any of claims 1-31 in combination with domestic water supply in a building for heating the water, for reducing the microbial count of the water, or for reducing a limescale developing effect of the water. Due to the size and installation requirements it is possible to place the device under the sink or as close to the faucet as desired.

In one embodiment the device may be used as a kitchen tap that produces instant boiling water for household purpose, e.g. for tea or coffee making. The device reduces limescale and eliminates the dry fire risk known from a traditional resistive heating element. In addition, the device has no water loss as in a kettle or unnecessary power consumption as in known consumer products and has an efficiency of 100% except for loss to wiring and cables. Further, the device is able to be both tank-less or suited with a tank, the tank will though reduce the efficiency. The device is highly scalable within flow, temperature, power consumption, volts/amps, phases and size. Even further, the device hydrogen enriches the water. In one embodiment the device may be used for space heating in e.g. residential or commercial buildings. The device uses AC current in an open or closed water circulation system with a very high efficiency, 100%. There is no limescale or chance of dry fire of heat element in the device. The device produces instant heating with temperature ratio from inlet temperature to boiling point. Further, the device is highly scalable within flow, temperature, power consumption, volts/amps, phases and size, and allows the use of renewable energy. Even further, the device is suitable as a booster to current water solution space heating.

In an twelfth aspect, the invention provides a method for reducing the microbial count of a liquid medium, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the liquid medium between the two electrodes such that the liquid medium is exposed to an electrical field based on the AC electrical signal until the microbial count is reduced.

The liquid medium may e.g. be a body fluid, a beverage, or waste water. The method may e.g. be carried out internally in a device configured for blood transfusion or in a device configured for waste water treatment.

In a thirteenth aspect, the invention provides a body fluid or a beverage or a liquid medium for washing purpose and which has been treated according to the method of the eleventh aspect.

In a fourteenth aspect, the invention provides a method for increasing the content of hydrogen in a liquid medium, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the liquid medium between the two electrodes such that the liquid medium is exposed to an electrical field based on the AC electrical signal until the content of hydrogen in the liquid medium is increased.

The liquid medium may e.g. be a body fluid, a beverage, or waste water. The method may e.g. be carried out internally in a device configured for blood transfusion or in a device configured for waste water treatment.

In a fifteenths aspect, the invention provides a beverage which has been treated according to the method of the thirteenth aspect of the invention. The method may e.g. be carried out internally in a device selected from the group consisting of: coffee machines, water coolers, ice makers, beer taps, soda water taps, and orange juice taps.

In a sixteenths aspect, the invention provides a method for decreasing a limescale developing effect of a liquid medium, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the liquid medium between the two electrodes such that the liquid medium is exposed to an electrical field based on the AC electrical signal until the limescale developing effect of the liquid medium is reduced.

The method may e.g. be carried out internally in a device selected from the group consisting of: high pressure washers, dish washers, washing machines, window washers, car washers, air plane washers, sprinkling systems for cars, sprinkling systems for fire extinguishing, lawn and garden sprinkling systems.

In a seventeenths aspect, the invention provides a liquid medium treated by the method according to the fifteenths aspect.

In an eighteenths aspect, the invention provides a method for treating water in a mobile application selected from a group consisting of: cars, airplanes, boats and caravans, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the water between the two electrodes such that the water is exposed to an electrical field based on the AC electrical signal until the temperature of the water is increased, or until the content of hydrogen in the liquid medium is increased or until the microbial count in the water is reduced.

In a nineteenths aspect, the invention provides a method for boosting temperature of a fluid medium in a green energy application selected from the group consisting of: a solar power application, a geothermal heating application, and a district heating application, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the fluid medium between the two electrodes such that the fluid medium is exposed to an electrical field based on the AC electrical signal until the temperature of the fluid medium is increased.

The booster will assist in keeping a steady and/or constant temperature in a liquid no matter the inlet temperature. The device being 100% efficient except for loss to wiring and cables, free of limescale, no chance for dry fire of heat element, able to use renewable energy, able to use power directly from the grid and highly scalable within flow, temperature, power consumption, volts/amps, phases and size. Further the device reduces bacteria count when treating the liquid and enriches the hydrogen content in the water. In addition, the device is able to use renewable energy since it will work with relatively large variations in voltage and amps.

In a twentieth aspect, the invention provides a method for reducing half-life of a radioactive fluid substance, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the fluid medium between the two electrodes such that the fluid medium is exposed to an electrical field based on the AC electrical signal until the half-life is reduced.

In a twenty-first aspect, the invention provides a method for treating waste water in a waste water treatment plant, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the waste water between the two electrodes such that the waste water is exposed to an electrical field based on the AC electrical signal until the temperature of the water is increased, or until the content of hydrogen in the liquid medium is increased or until the microbial count in the water is reduced.

In a twenty-second aspect, the invention provides a method for cleaning teat-cups or the udder of an animal with a liquid medium in a device for milking animals, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the liquid medium between the two electrodes such that the liquid medium is exposed to an electrical field based on the AC electrical signal until the temperature of the water is increased, or until the content of hydrogen in the liquid medium is increased, or until the microbial count in the water is reduced.

In a twenty-third aspect, the invention provides a method for improving the freshness or visual appearance of fruit or vegetables by sprinkling the fruit or vegetable with a liquid medium, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the liquid medium between the two electrodes such that the liquid medium is exposed to an electrical field based on the AC electrical signal until the temperature of the water is increased, or until the content of hydrogen in the liquid medium is increased, or until the microbial count in the water is reduced.

In a twenty-fourth aspect, the invention provides a method for cleaning parts of a cooling device with a liquid medium, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the liquid medium between the two electrodes such that the liquid medium is exposed to an electrical field based on the AC electrical signal until the temperature of the water is increased, or until the content of hydrogen in the liquid medium is increased, or until the microbial count in the water is reduced. In one embodiment, the device may be used to remove or lower the count of legionella in cooling towers. Legionella occurs in cooling towers where hot water is cooled by air or water leaving a perfect temperature for legionella growth. Any drift or water leaving the cooling tower by way of the air, can cause an outbreak of Legionnaires disease. A range of ways of disinfection or removing of legionella has been outlined but they all rely on chemicals and human interaction, thus creating a possibility for infection. The device according to the invention may be used for treatment of condensed water from a cooling device or for preparing a liquid medium for washing the condenser, the evaporator and other parts of a cooling device.

In a twenty-fifth aspect, the invention provides a method for more generally amending a liquid medium by electrical treatment. According to this method, both the limescale developing ion and the microbial count is reduced in one and the same treatment. This method comprises providing a first and a second electrode, connecting the electrodes to an AC power supply which provides an AC electrical signal, operating the AC power supply to provide an electrical potential between the electrodes which potential varies with an AC electrical signal, and establishing a flow of the liquid medium between the two electrodes such that the liquid medium is exposed to an electrical field based on the AC electrical signal until the limescale developing effect of the liquid medium is reduced and until the microbial count in the liquid medium is reduce.

In a twenty-sixth aspect, the invention provides a method for reducing salt concentration in salt water, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the salt water between the two electrodes such that the salt water is exposed to an electrical field based on the AC electrical signal until concentration of salt in the saltwater is reduced.

In a twenty-seventh aspect, the invention provides a method for treating water in an aquarium, the method comprising providing a first and a second electrode, connecting the electrodes to an AC power supply which provides an AC electrical signal, operating the AC power supply to provide an electrical potential between the electrodes which potential varies with an AC electrical signal, and establishing a flow of the aquarium water between the two electrodes such that the aquarium water is exposed to an electrical field based on the AC electrical signal until concentration of salt in the aquarium water is reduced, or until the temperature of the aquarium water is increased, or until the microbial count in the aquarium water is reduced, or until the content of hydrogen in the aquarium water is increased.

Additional Examples of Applicability

In one embodiment the device may be used for clean water supply for residential properties, in industry e.g. for treatment of process water, in office buildings, in hospitals, for treating the fluid medium in various tanks, e.g. in marine tanks etc.

The device gives a continuous flow of cleaned water which is instantly treated without use of chemicals etc. and if desirable without or essentially without an increase in temperature.

There will be no limescale in the following pipe installation due to decomposition of limestone developing elements in the liquid medium. Further the device serves Easy Build-in and is highly scalable within flow, temperature, power consumption, volts/amps, phases and size. In addition, the device hydrogen enriches the water and is able to make hot water as well. Even further, the device is able to use renewable energy.

In one embodiment the device may be used for dentist water and similar. The device produces tempered water with reduced bacteria count or even disinfected water, thus the patient will get clean water into the mouth during treatment. In addition, the device hydrogen enriches the water. Further, the device is highly scalable within flow, temperature, power consumption, volts/amps, phases and size. Even further, the device is able to use renewable energy.

In one embodiment the device may be used in an electric kettle. The kettle offers 100% efficiency except for loss to wiring and cables, no limescale, able to make boiling water with e.g. less than 1 kW, faster hot water, no dry fire of heat element and will be highly scalable within flow, temperature, power consumption, volts/amps, phases and size. Further, the device hydrogen enriches the water. In addition, the device is able to use renewable energy.

In one embodiment the device may be used for producing what herein will be referred to as “medico water”, i.e. water treated in accordance with the present invention. The device instantly produce high amount of strong antioxidants in liquids containing H2O due to hydrogen enrichment. For diabetes patients it may be an advantage to drink medico water and with an improved result. Medico water may cure or treat other illnesses; Superoxide in brain, Neonatal brain hypoxia, Restraint-induced dementia, Alzheimer's disease, Senile dementia, Parkinson's disease, Spinal cord injury, Glaucoma eye, Corneal alkali-burn eye, Hearing disturbance, Lung cancer, Oxygen-induced lung injury, Myocardial infarction heart, Irradiation-induced heart injury, Obstructive jaundice liver, Cisplatin nephropathy kidney, Kidney transplantation kidney, Acute pancreatitis, Intestines, Atherosclerosis blood vessel, Diabetes mellitus type 2, Metabolic syndrome, Obesity/Diabetes, Tongue carcinoma cancer, Allergy type I and Radiation injury. Further, it may offer instant production and is able to produce hot or tempered water. Even further, the device is highly scalable within flow, temperature, power consumption, volts/amps, phases, and size.

In one embodiment the device may be used at MW facilities, i.e. for large-scale treatment of liquid substances. The unit is able to produce several MW of heat instantly in a continuously flow of liquid and with an efficiency close to 100%. The unit does not need a heat exchanger, but can treat the liquid medium directly. The unit is able to regulate the production within seconds, which makes it possible to convert overproduction of electricity into thermal energy which can be sold or stored for later use. Further, the device offers no limescale, no dry fire of heat element, reducing limestone due to decomposition hereof and a cleaning ability that reduces bacteria count. Even further, the device is highly scalable within flow, temperature, power consumption, volts/amps and phases.

In one embodiment the device may be used for process water e.g. meat industry, textile industry, soap etc. The device highly reduces bacteria count without highly increasing the temperature of the liquid and highly reduces limestone and limescale due to decomposition of limescale developing elements in the liquid medium. In addition the device may increase the content of hydrogen in the liquid medium. The device enables an increased washing effect due to hydrogen enrichment and softening of the water. Further, the device is able to produce hot water with efficiency of 100% except for loss to wiring and cables. Even further, the device is highly scalable within flow, temperature, power consumption, volts/amps, phases and size. In addition, the device is able to use renewable energy.

In one embodiment the device may be used as a steamer e.g. for use in coffee machines, for ironing or for any steam production. The device provides very fast production of steam with an infinitely temperature rise from inlet temperature to boiling point and with a 100% efficiency except for loss to wiring and cables. In addition, the steam will be hydrogen enriched. The device will not build up limescale or limestone due to decomposition hereof and it will not experience any dry fire of heat element. Further, the device is able to use renewable energy and able to produce steam with little electricity and use the power of steam expanding. Even further, the device is highly scalable within flow, temperature, power consumption, volts/amps, phases and size.

In one embodiment the device may be used in swimming pools, hot tubs, spas etc. The device is an electric unit using AC to heat and clean water. The device gives a continuous flow of hot and/or cleaned water without the use of chemicals such as chloride. There is no need of special bacteria filters and chloride resistant bacteria can be removed e.g. Crypto Sporidum. The device eliminates bacteria in the water circulation system. Further, the device will not build up limescale or limestone due to decomposition hereof and it will not experience any dry fire of heat element. In addition, the device hydrogen enriches the water. Even further, the device is highly scalable within flow, temperature, power consumption, volts/amps, phases and size. The device is able to use renewable energy.

In one embodiment the device may be used as an UN disaster unit for use in disaster areas. The device reduces microbial count in a continuous flow of water without highly increasing the temperature and without the use of chemicals, but only by use of AC current. The device is able to use renewable energy and is also able to produce hot water. Further, the device is highly scalable within flow, temperature, power consumption, volts/amps, phases and size, and the treated water is hydrogen enriched. Even further, the device will not build up limescale or limestone due to decomposition hereof and it will not experience any dry fire of heat element.

In one embodiment of the device nitrate filled water is cleaned from nitrate thus water wells at farm areas are improved for drinking water facility without treating the water in a costly way. Further, the device hydrogen enriches the water and is highly scalable within flow, temperature, power consumption, volts/amps, phases and size.

In one embodiment of the device, oil can be treated, thus removing bacteria and impurities which results in an improved oil of better quality. Further, the oil will be heated for even better viscosity. Even further, the device is highly scalable within flow, temperature, power consumption, volts/amps, phases and size.

In summary, the device may be applied for residential heating, district heating, mobile electric radiators, booster in connection with geothermal heating or solar heating, tank-less water heater, faucet, steam appliances, heating units for e.g. dishwashers, washing machines, unit kettles, instant hot water dispenser, car heating, iron, dryer, cleaning of e.g. tap water, marine tank liquid, swimming pools, Jacuzzis, spa baths, cooling towers, faucets, treatment in relation to legionella, and other cleaning measures such as sewage cleaning, medico cleaning units, dentist water dispenser, water aid at disaster areas or in developing countries, coffee machine, atomizer, hydrogen production, hydrogen enrichment, decontamination, storage of electricity, power plants, process water for industry use, treatment of nitrate, pasteurization, sterilisation disinfection, treatment of oil, growth improvement of plants, vegetables, animals, muscles etc. E.g. in connection with washing machines may facilitate heating without or at least with a reduced deposition of sediments from washing powder, dirt and/or water.

### BRIEF DESCRIPTION OF THE DRAWINGS

Embodiments of the invention will now be further described with reference to the drawings, in which:

FIG. 1 illustrates an instant type liquid treating device according to the invention,

FIGS. 2 and 3 illustrate different views of the device of FIG. 1

FIG. 4 illustrates another embodiment of an instant type liquid treating device according to the invention,

FIG. 5 illustrates a conical outer channel for the embodiment of FIG. 4,

FIGS. 6 and 7 illustrate difference elements of for the embodiment of FIG. 4;

FIG. 8 illustrates an example of an embodiment for a blood treatment device;

FIG. 9 illustrates an example of a device for instant treatment of a liquid for boats and caravans;

FIG. 10 illustrates an example of a booster for e.g. Solar-, geothermal- or district heating, with an inlet, outlet and a housing;

FIG. 11 illustrates an example of the installation for a device treating supply water or water from marine tanks;

FIG. 12 illustrates an illustration of a coffee machine;

FIG. 13 illustrates an illustration of a device for producing dentist water and a handle for the doctor to pour the water into the patient's mouth;

FIG. 14 illustrates an example of a dishwasher with the device inside the dishwasher with an inlet and an outlet;

FIG. 15 illustrates an illustration of an electric kettle;

FIG. 16 illustrates an example of a device for instantly killing Legionella bacteria;

FIG. 17 illustrates an example of a device for instant hydrogen enrichment of water;

FIG. 18 illustrates an example of a device for mobile space heating;

FIG. 19 illustrates an example of a mobile water heater;

FIG. 20 illustrates an example of a device for instant production of several MW heat in a liquid;

FIG. 21 illustrates an example of a device for removing nitrate in water;

FIG. 22 illustrates an example of a device for treating oil;

FIG. 23. Illustrates an example of a device to treat wastewater, plant water, radioactive liquids, process water, pasteurization of juice, milk or any beverages and for treating water used to spray upon fruit and vegetables or dirty water in e.g. disaster areas;

FIG. 24 illustrates an example of a device for instant boiling water;

FIG. 25 illustrates an example of a device for space heating;

FIG. 26 illustrates an example of a device for producing steam to e.g. ironing, coffee machines etc;

FIG. 27 Illustrates an example of a swimming pool;

FIG. 28 illustrates an example of a tankless water heater;

FIG. 29 illustrates an example of a washing machine;

FIG. 30 illustrates an example of a water dispenser with a body, a replaceable water tank and a fixed water inlet;

FIG. 31 illustrates an example of a 3 phased unit with a telescopic layout;

FIG. 32 and FIG. 33 illustrates a cross view of the telescope and outer electrodes;

FIG. 34 illustrates the shoulder and recess in the telescopic layout; and

FIG. 35 illustrates the electrical wiring for a device. The device is able to use 1, 2 or 3 phases with simple wiring in the connection box.

### DETAILED DESCRIPTION OF EMBODIMENTS

It should be understood that the detailed description and specific examples, while indicating embodiments of the invention, are given by way of illustration only, since various changes and modifications within the spirit and scope of the invention will become apparent to those skilled in the art from this detailed description.

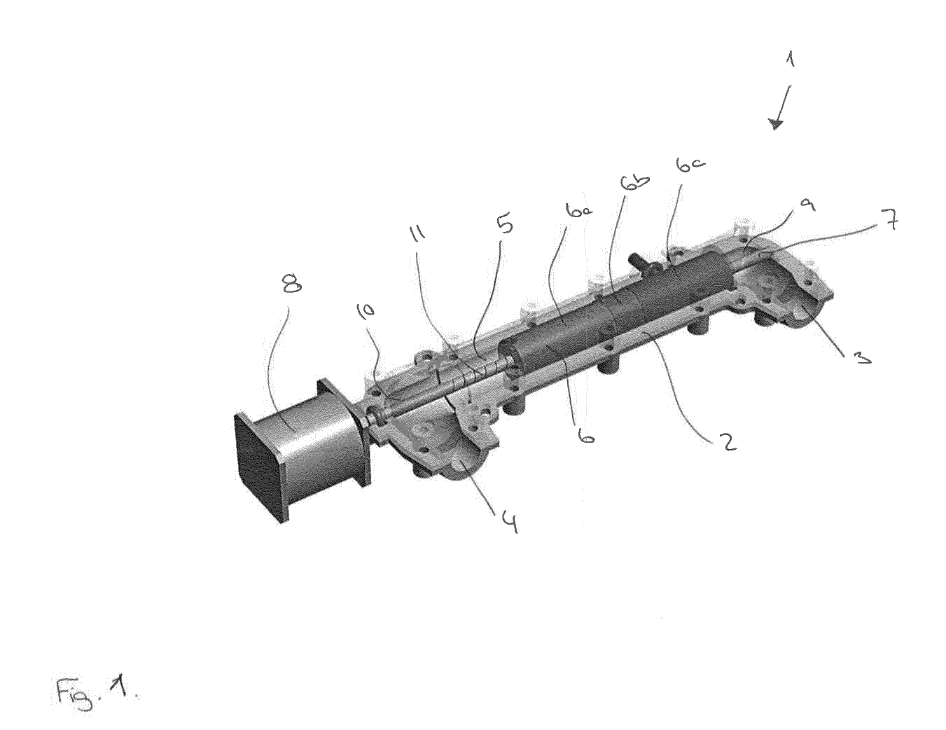

FIG. 1 illustrates a part of an embodiment of an instant type liquid treating device 1 which comprises a housing 2 with an inlet 3 and an outlet 4. Only one half of the housing is illustrated as the upper part is removed to be able to illustrate the inner of the housing.

The inlet 3 and the outlet 4 are connected by a passage 5 for passing a liquid medium through the device 1. The device further comprises a set of cooperating separate conductive faces 6, 7 which are connectable to an AC-power supply. The conductive faces 6, 7 are directly exposed to each other in the passage 5 such that the liquid medium can be treated by way of its own electrical resistance between the faces 6, 7.

A control 8 facilitates relative movement of the faces 6, 7. This unit 8 comprises a servo motor, and control means for operating the motor e.g. based on e.g. current, voltage, conductivity of the liquid, liquid temperature, liquid hardness, and liquid quality. Thus, the control 8 facilitates relative movement of the faces 6, 7, and thereby enables changing of the electrical potential between the faces, whereby the treatment of the liquid can be adapted to a specific environment and thus type of liquid, e.g. quality of the liquid.

In the present embodiment, the inlet 3 and outlet 4 are positioned transverse to the passage 5. The transitions 9, 10 between the inlet 3 and the passage 5 and the outlet 4 and the passage 5 are formed with rounded corners to minimise influence on the liquid flow internal in the passage 5.

In the present embodiment, the conductive faces 6, 7 are parallel to each other and thus parallel to a flow-path formed between the conductive faces.

The conductive faces 6, 7 are formed as cylindrical electrodes such that an outer one of the electrodes 6 forms a channel in which the other, inner, electrode is received 7. Thus, the outer electrode 6 is arranged as a cylinder around the inner electrode 7 which is formed as a cylinder having solid core.

In the illustrated embodiment, the outer electrode 6 comprises three individual cylindrical elements 6a, 6b, 6c axially movable relative to each other. Thus, the three individual cylindrical elements 6a, 6b, 6c are arranged in a telescope-like arrangement, thereby enabling relative movement of the outer electrode 6 and the inner electrode 7 relative to each other by changing the effective length of the outer electrode 6.

In the present embodiment, the inner electrode 7 is fixed to the housing 2 by a non-conductive element, whereas the outer electrode 6 is movable by used of the rotating spindle 11. To avoid current leakage, the spindle 11 is electrically isolated from the outer electrode 6 by use of a fastening element not shown made from a non-conductive material.

Thus, the relative movement of the faces 6, 7 is provided by movement of the three individual cylindrical elements 6a, 6b, 6c relative to each other.

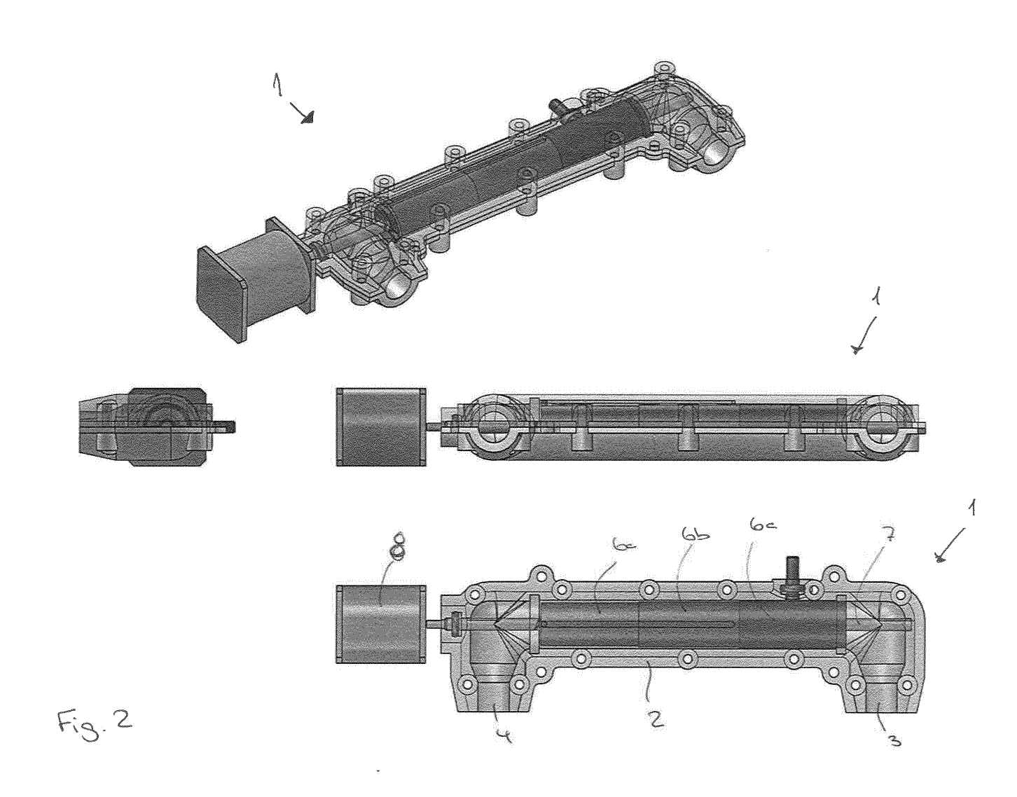

FIGS. 2 and 3 illustrate different views of parts of an embodiment of an instant type liquid treating device 1 of FIG. 1.

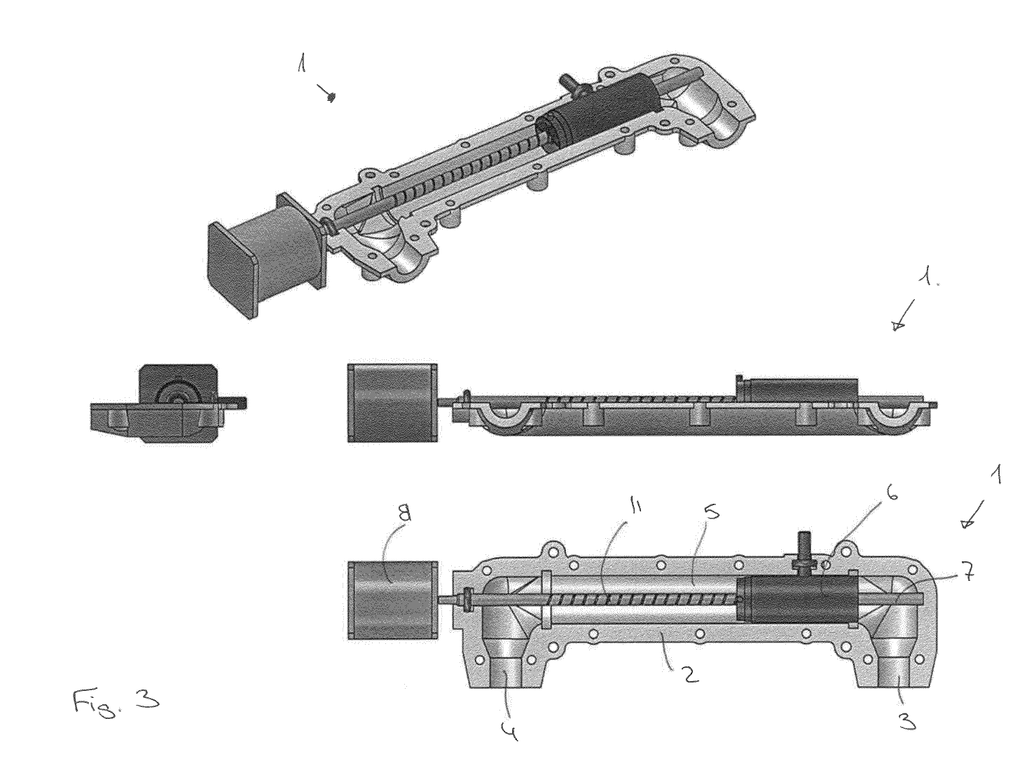

FIG. 4 illustrates a part of an embodiment of an instant type liquid treating device 101 which comprises a housing 102 with an inlet 103 and an outlet 104. Only one half of the housing is illustrated as the upper part is removed to be able to illustrate the inner of the housing.

The inlet 103 and the outlet 104 are connected by a passage 105 for passing a liquid medium through the device 101. The device further comprises a set of cooperating separate conductive faces 106, 107 which are connectable to an AC-power supply. The conductive faces 106, 107 are directly exposed to each other in the passage 105 such that the liquid medium can be treated by way of its own electrical resistance between the faces 106, 107.

A control not shown facilitates relative movement of the faces 106, 107. This unit may be identical to the control 8 of FIG. 1. The control facilitates relative movement of the faces 106, 107, and thereby enables changing of the electrical potential between the faces, whereby the treatment of the liquid can be adapted to a specific environment and thus type of liquid, e.g. quality of the liquid.

In the present embodiment, the conductive faces 106, 107 are parallel to each other and thus parallel to a flow-path formed between the conductive faces.

The conductive faces 106, 107 are formed as cylindrical electrodes such that an outer one of the electrodes 106 forms a channel in which the other, inner, electrode is received 107. Thus, the outer electrode 6 is arranged as a cylinder around the inner electrode 7 which is formed as a cylinder having solid core.

In order to facilitate a simultaneous change in an amount of confronting areas of the faces 106 and 107 and the spacing between the confronting faces, the outer electrode 106 comprises a surface portion which is non-parallel to at least a portion of the other face 107. When the conductive faces 106, 107 are shifted relative to each other in the passage 105, the distance between the conductive faces will vary along the effective flow-path due to the oblige angle.

Thus, in the illustrated embodiment, a surface portion 106a of the outer conductive face 106 is provided as a conical section. FIG. 5 illustrates a conductive face 106 which is usable as an outer electrode, the outer electrode 106 comprising a conical section 106a and a cylindrical section 106b.

The outer electrode 106 is arranged so that it can be shifted axially in the passage 105, thereby allowing for relative movement of the conductive faces 106, 107. The outer electrode 106 is movable by used of the rotating spindle 111. To avoid current leakage, the spindle 111 is electrically isolated from the outer electrode 106 by use of a fastening element 112 made from a non-conductive material.

FIGS. 5, 6, and 7 illustrate different views of parts of an embodiment of an instant type liquid treating device 1 of FIG. 1.

FIG. 5 illustrates a conductive face 106 which is usable as an outer electrode. The outer electrode 106 comprises a conical section 106a and a cylindrical section 106b. The cylindrical section 106b is provided with a pair of through holes 113 for attachment of the fastening element 112 illustrated in FIG. 4.

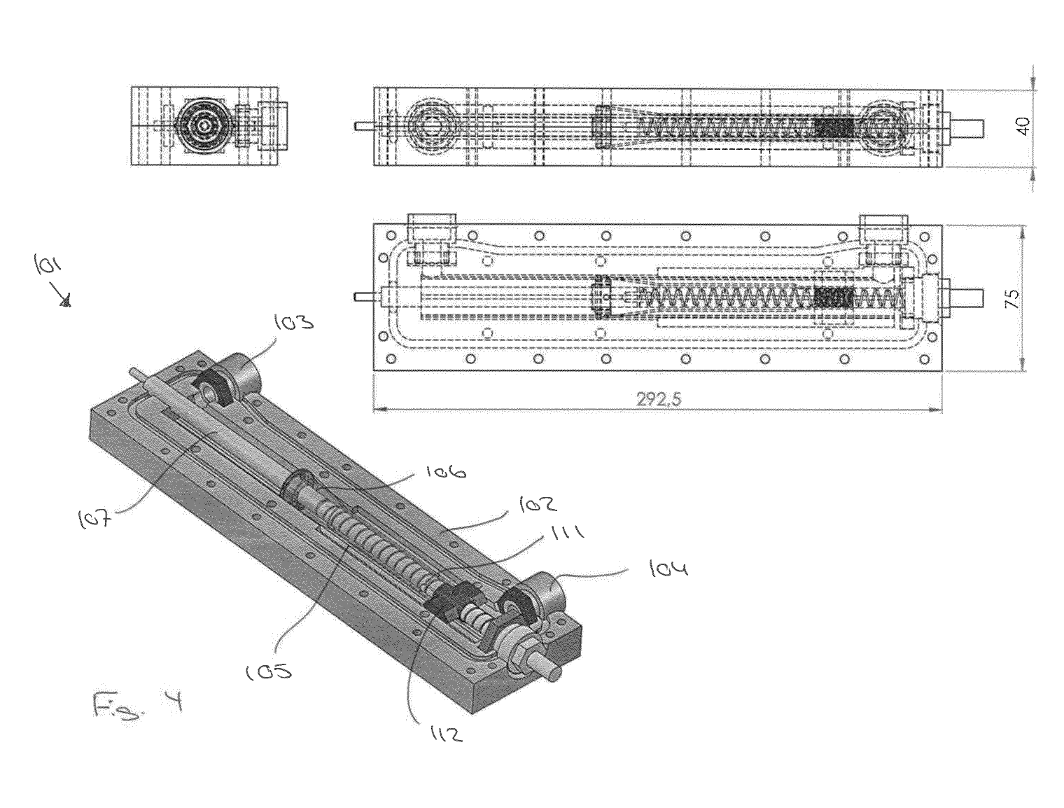

FIGS. 6 and 7 illustrate different views of the spindle 111 and the inner electrode 107. In the embodiment illustrated in FIG. 7, the spindle 111 and the inner electrode 107 are positioned in the housing 102′. The inlet 103′ is positioned at the end of the housing 102, whereas the outlet 104 is positioned transverse to the passage 105 as in the embodiment of FIG. 4.

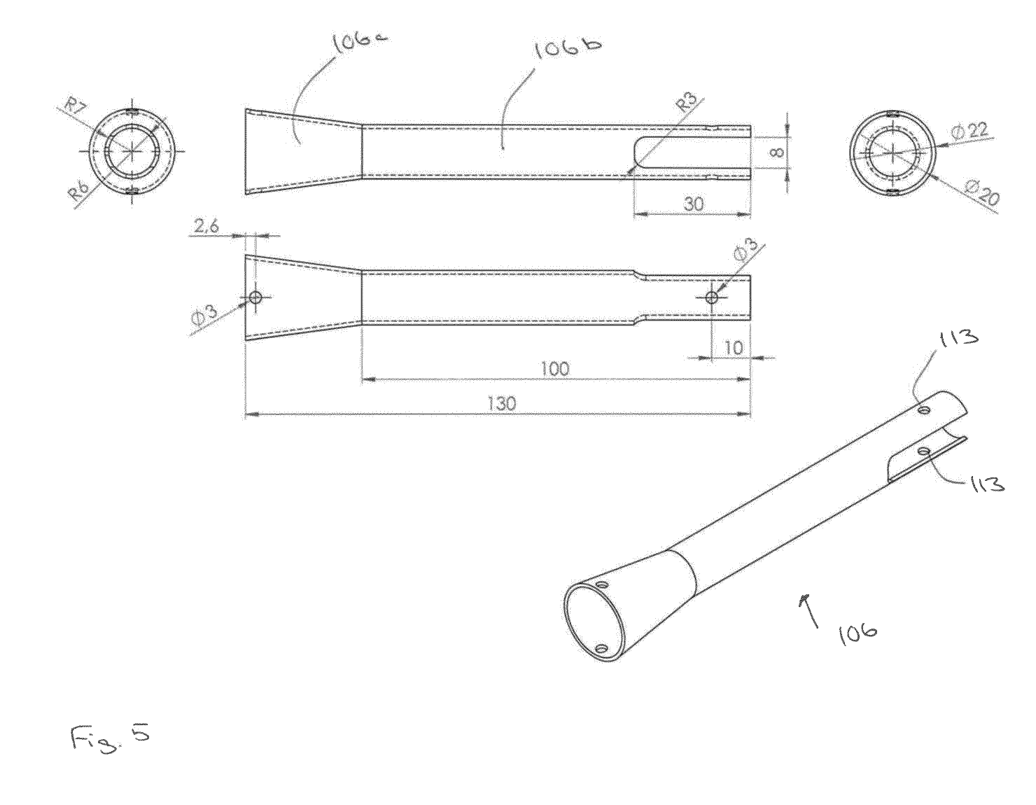

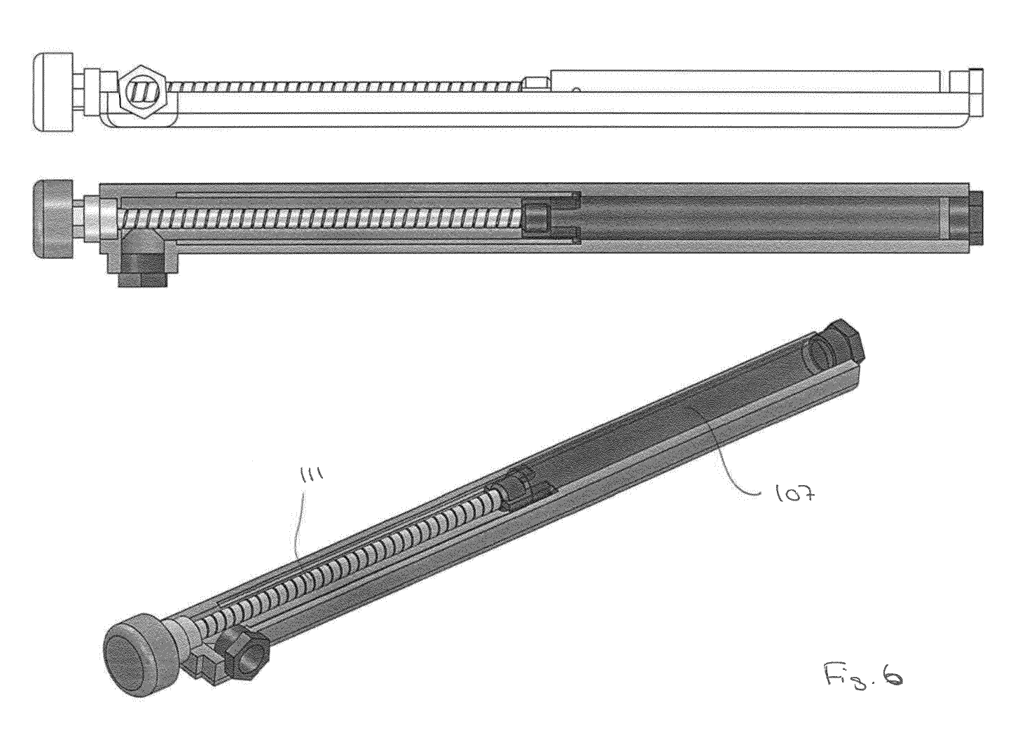

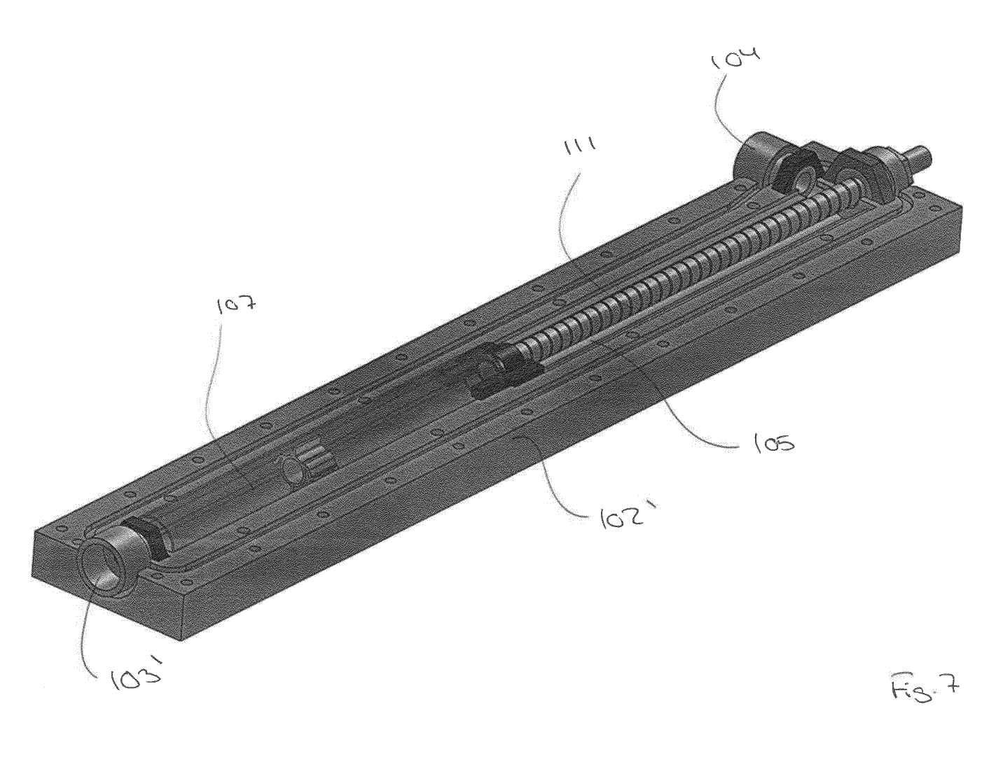

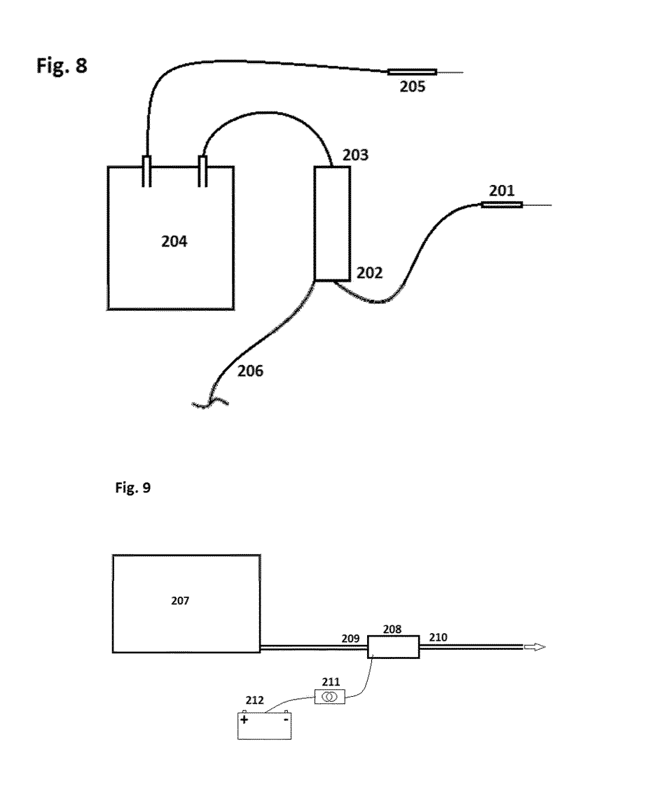

The illustration on FIG. 8 shows a needle 201 for tapping blood from a patient. From the needle the blood is led to the inlet 202 of the treating device wherein the blood will be treated for bacteria. From the outlet 203 of the device the treated blood is led to a receiving bag 204 wherefrom the blood can go back to the patient trough a needle 205 when the bag is filled or when enough blood has been treated. The device is suited with an extension cord 206.

FIG. 9 shows a water tank 207 and the device 208 for boats and caravans to instantly treat a liquid. A fixed pipe is arranged between the water tank 207 and the inlet 209 on the device 208. From the outlet 210 on the device, a pipe extends to the consumer. The electric power is arranged from a battery 212 through a converter 211 to the device 8. The converter 211 lowers the amps and raises the volts making it easier for electric wiring.

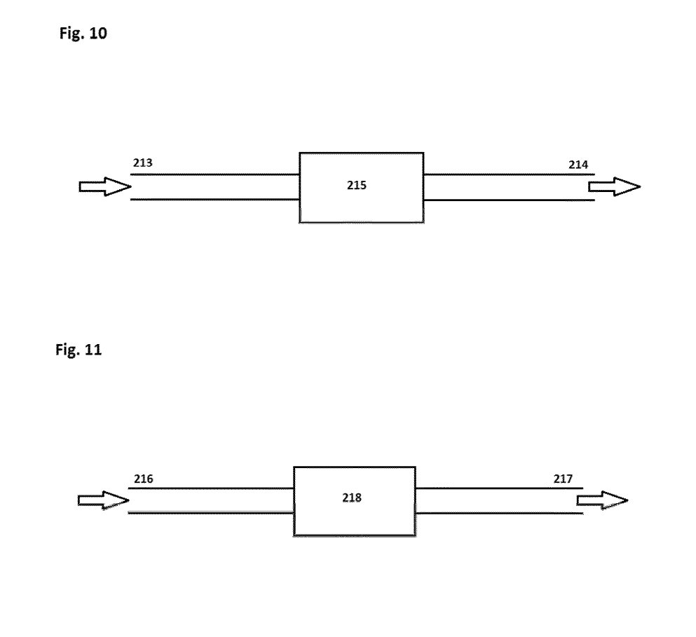

FIG. 10 represent an inlet 213 and outlet 214 performing a flow path with an housing 215 for the device.

The illustration on FIG. 11 shows an inlet 216 for the supply water or the water from a marine tank/ballast tank, an outlet 217 for the treated liquid and a housing 218 where the treatment takes place.

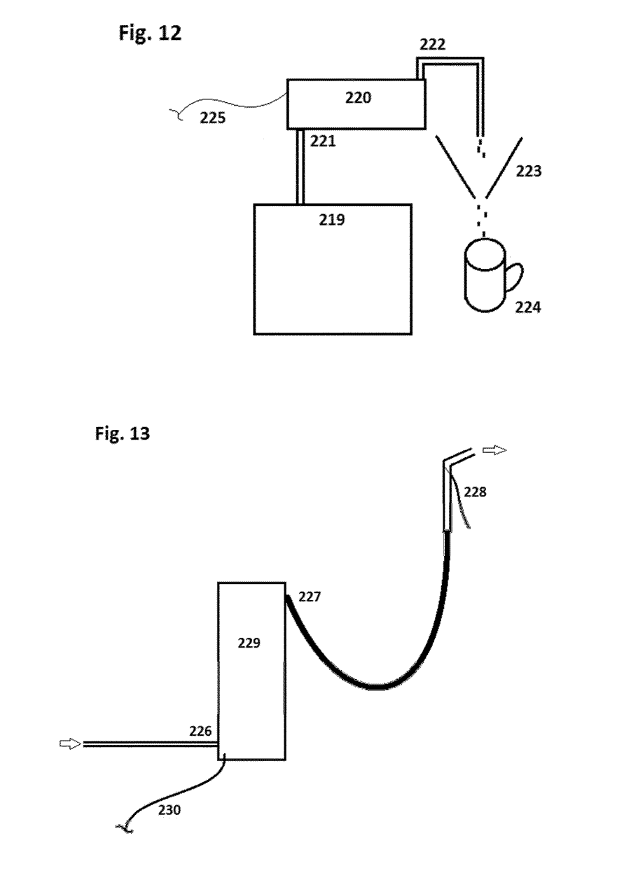

The illustration on FIG. 12 shows a water tank 219 from where the water is led to the inlet 220 of the housing 221 wherein the device for heating, hydrogen enrichment and cleaning of the water is placed. From the outlet 222 of the device, the treated water is led to a funnel 223 where it pours down upon the grounded coffee beans so that coffee will be produced and pour down to a reservoir e.g. a cup 224. The coffee machine is suited with an extension cord 225.

The illustration on FIG. 13 shows a device 229 for tempered dentist water production. The device 229 has an inlet 226 and an outlet 227 for water to pass through. From the outlet 227 the treated water is led to a handle 228 for a user to control the amount of water used. The device is suited with an extension cord 230.

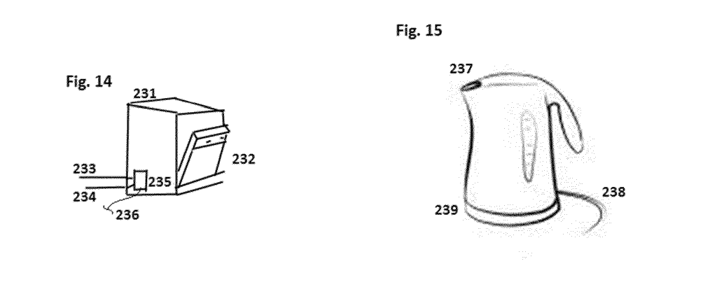

The illustration on FIG. 14 illustrates a dishwasher 231 comprising a room for dishes, a door 232 with control panel and the device 235 for water treatment. The device 235 consists of an inlet 233 and outlet 234 performing a flow path for the water to be treated. Further the device is suited with an extension cord 236.

The illustration of an electric kettle shown on FIG. 15 illustrates an inlet 237 and outlet 237 for a kettle. The kettle then has a docking station 239 for electric connection from the electric cord 238.

The illustration on FIG. 16 illustrates an inlet 240 for the legionella contaminated liquid to enter the device 241 wherein the treatment of the liquid takes place. After the instant treatment the cleaned liquid exits the device 241 through the outlet 242 of the device 241.

The illustration on FIG. 17 illustrates an inlet 243 for water to enter the device 244 wherein the water will be cleaned for bacteria and also be hydrogen enriched. After the treatment the cleaned and hydrogen enriched water will exit the device through the outlet 245.

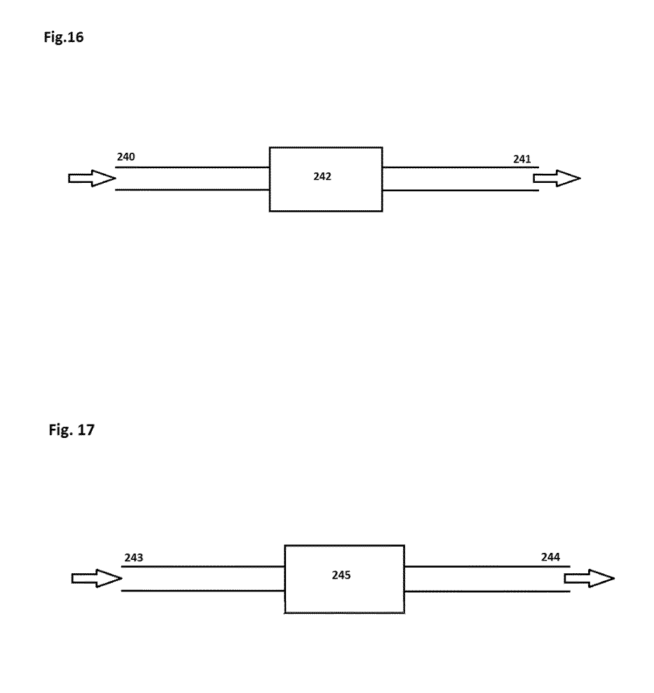

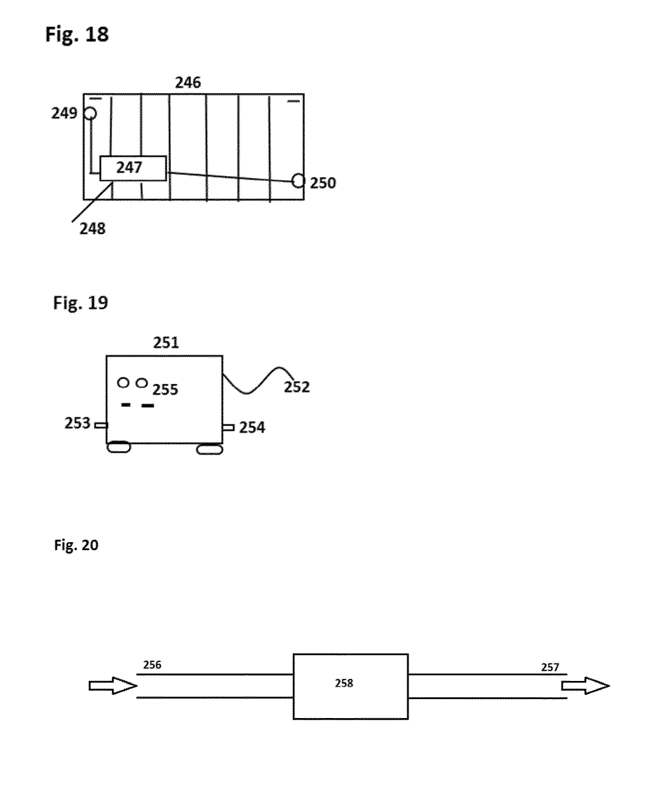

The illustration on FIG. 18 illustrates a radiator 246 with an instant heating device 247 that will instantly heat a liquid in the closed loop of the radiator. The heating device I placed between the inlet 249 and outlet 250 of the radiator, thereby creating a closed circulation loop. The radiator can be wall mounted and unite with the surroundings or free up some floor space in e.g. a house. The radiator only needs electricity be the extension cord 248.

The illustration on FIG. 19 illustrates a mobile water heater 251 with an inlet 253 for e.g. water to enter the device wherein the water will be treated, after the treatment the water will exit the device through the outlet 254. The mobile water heater needs water connected to the inlet 253 and electricity connected through the extension cord 252. The device has a control panel 255 for easy use.

The illustration on FIG. 20 illustrates a device 256 for instant production of several MW heat in a liquid running through from the inlet 257 of the device to the outlet 258 of the device.

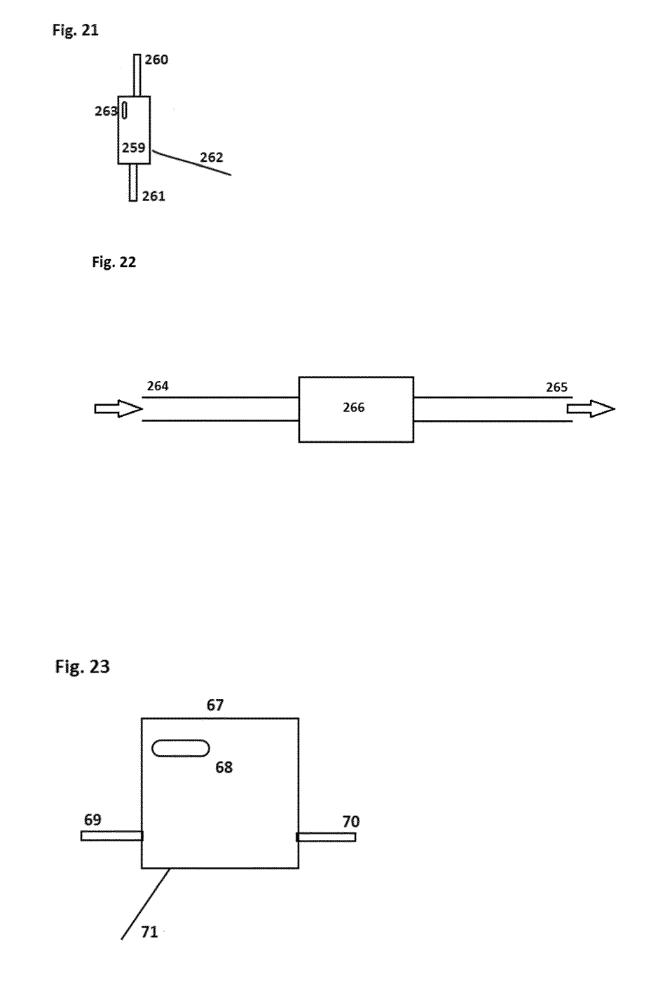

The illustration on FIG. 21 illustrates a device 259 with an inlet 261 for nitrate contaminated water to enter the device wherein the nitrate will be removed from the water. After the treatment the cleaned water will exit the device through the outlet 260. The device is suited with an extension cord 262 and a control panel 263.

The illustration on FIG. 22 illustrates a device 266 wherein a treatment of oil takes place. The oil enters the device via the inlet 264 and exits the device through the outlet 265. The treatment is instant in a continuously flow of oil.

At FIG. 23 the device 67 has a control panel 68 an inlet 69 where the untreated liquid is pumped in and thereafter treated with the device 67 to finally leave as treated liquid out of outlet 70. The device is connected to a power supply via 71. Within 67 the liquid will pass through an electrical field, controlled by 68, where flow, temperature, watts and the unit can be controlled.

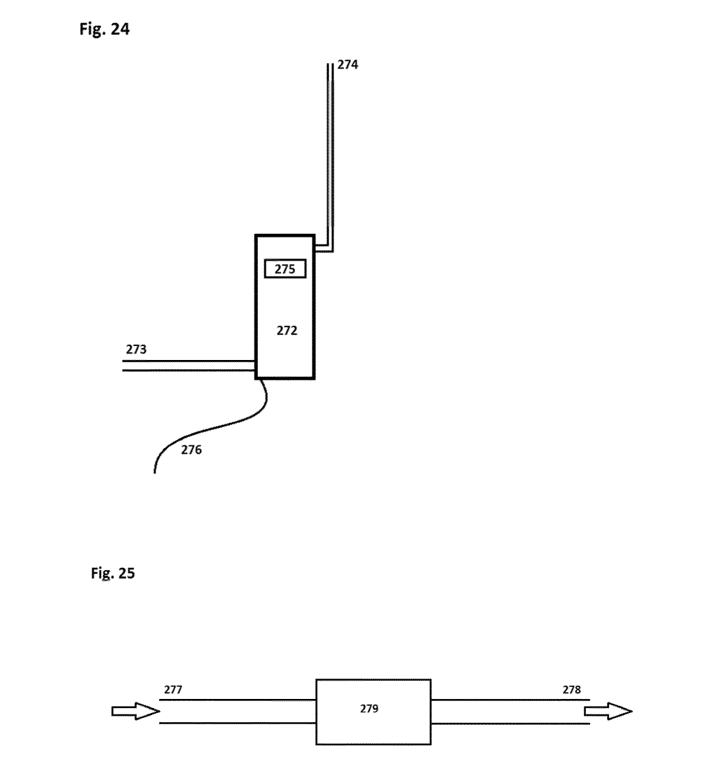

The illustration on FIG. 24 illustrates a device 272 for instant boiling water. The device 272 has an inlet 273 and an outlet 274 performing a flow path through the device 272. The device has an control panel 275 for e.g. setting temperature and flow. Further, the device will be powered by the extension cord 276.

The illustration on FIG. 25 illustrates a device 279 for heating a liquid for space heating. The device 279 comprises an inlet 277 and an outlet 278 forming a flow path for the liquid.

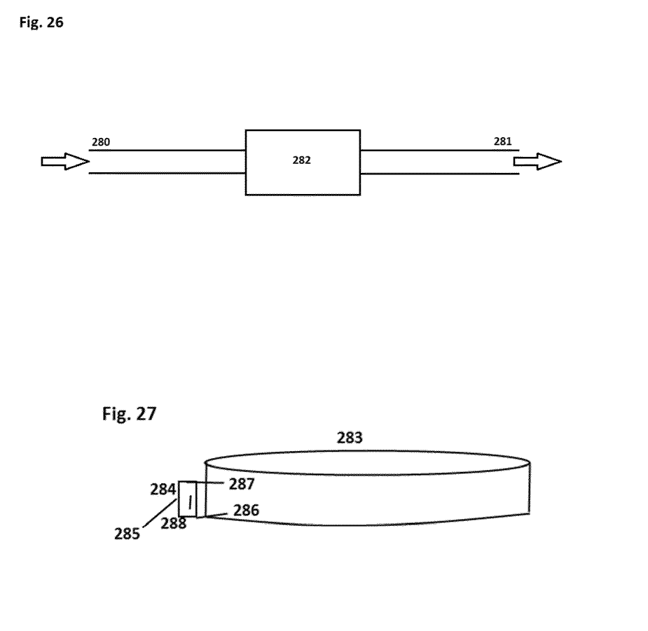

The illustration on FIG. 26 illustrates a device 282 comprising an inlet 280 and outlet 281 thereby forming a flow path for e.g. water to flow through and be treated.

FIG. 27 shows an example of a swimming pool 283 where the treatment device 284 is connected to a power supply 285 and where the device has an inlet 287 and an outlet 286. The water from the swimming pool or hot tub is pumped in to the inlet 287 treated by cleaning the water from bacteria alone or heated as well and then leaving through outlet 286. A control system 288 gives the user treatment options.

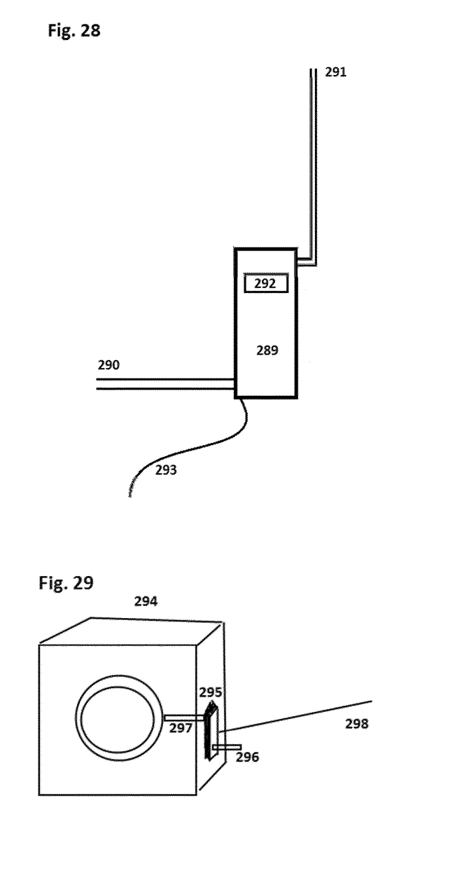

The illustration on FIG. 28 illustrates a device 289 comprising an inlet 290 for cold water to enter the apparatus to be treated and thereafter to exit through the outlet 291 to e.g. a faucet. The apparatus is powered by help of an extension cord 293 and is further suited with a control panel 292 for e.g. setting temperature and flow.

FIG. 29 shows a washing machine 294 that has a door for access to the washing drum, where dirty material is cleaned. The washing machine gets water via the inlet shows as 296 and which is treated in 295 and then delivered in to the drum via 297. The treatment core 295 is connected to power via 298. The washing machine further has a control panel for operating the machine.

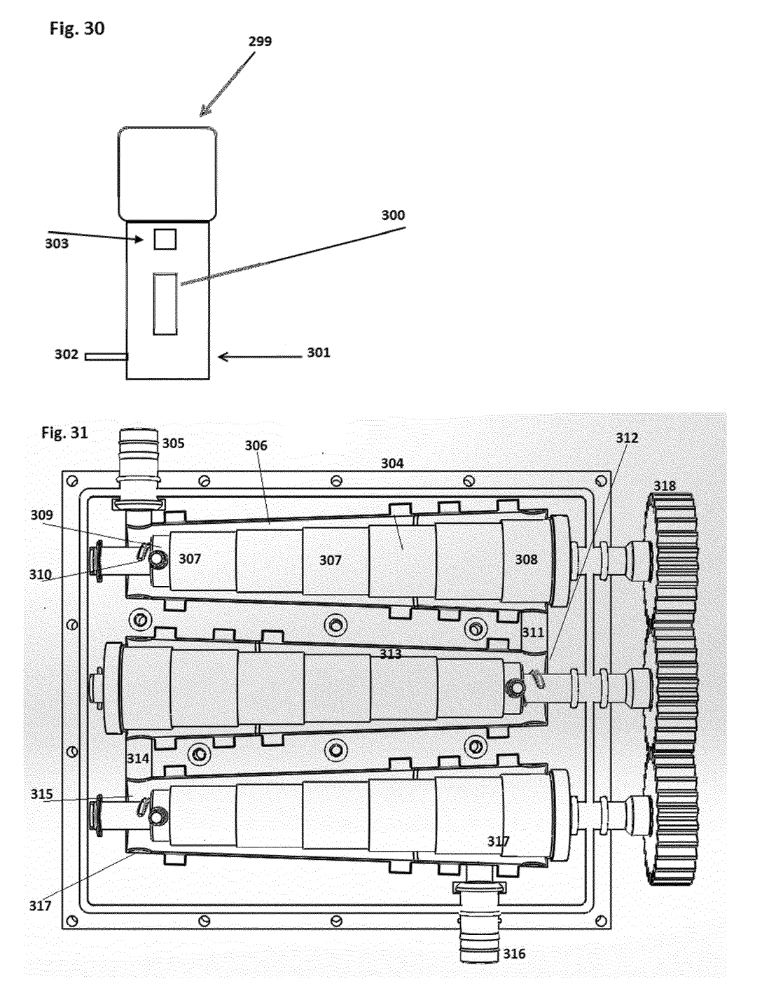

In FIG. 30 which show a water dispenser a replaceable tank of water 299 can be seen sitting on top of the body 301 that contains the heating and cleaning core 300 and where an inlet 302 is also present. In one product the water flows from the inlet 302 to the core 300 and out of the water dispenser outlet 303.

FIG. 31 shows an example of a 3 phase unit 304 in a cross section view. The liquid is let in via 305 into the first chamber, where the liquid will pass through an outer electrode 306 and an inner electrode 307 in a telescope like arrangement—here depicted with the electrode been made out of 6 parts, where the 308 greatest in size cylinder is attached to the unit and connect to the power supply. 309 is a nut with wings that is attached to the end part of the electrode 307 and that runs in a slitting which keeps the telescope in place while moving up and down the spindle that has thread 310. The spindle 310 connects to a handle or a gear 318 that can either connect to a motor or connect to other gears as show in the figure, that then connects to a single motor. The liquid flows between the electrodes 306 and 307 and out of the passageway 311 to tube no. 2, 312, where the liquid runs the opposite direction between further electrodes 313 and out of passageway 314 into the last tube or chamber 315. The chamber 315 is connected to the outlet 316 of the unit. 316 is placed in connection with the end part 317 of the telescope arranged electrode, thus the treatment takes place close to the outlet 316. The outer electrodes 306 is divided into parts that sits tight in a milled out 317 part of the unit 304, where the electrodes 306 in the one end by hatches connects to the milled out 317 part of the unit 304 and in the other end in held in places with a connector that goes through the unit 304 and is held in place with a nut. The electrodes 307 is show at full area, but 5 parts of the divided electrodes in a telescope like arrangement can fit in the 6th part 308 so that there Is a minimum of area in use. 308 can be used as a booster to enable even further effect area. The electrodes 306, 307 are arranged in a way where increasing area of the inner electrode brings the electrode closer to the outer electrode, while decreasing the area creates more space between the electrodes 306, 307.

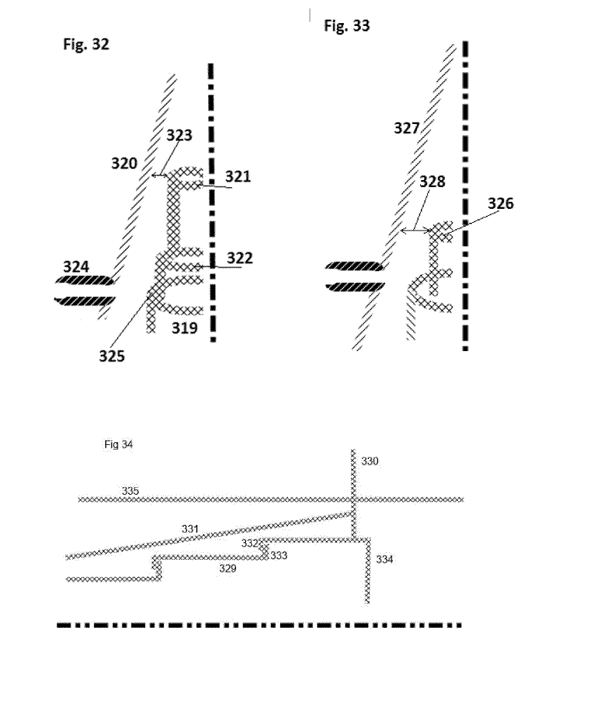

The FIGS. 32 and 33 shows a cross view of the telescope arranged electrode 319 and the outer electrode 320. In this arrangement the telescope arranged inner electrode b is increased first with one part 321 then with another part 322 and at the same time with area increase the electrodes are brought closer to each other 323 making the ideal setting for a stronger current to flow between the electrodes 319, 320. When the inner electrode 319 is decreased in area to lower the effect into the liquid that, flows between the electrodes, the space between the electrodes are at the same time increased to create the ideal setting for a weaker current to flow between the electrodes. The outlet shown as 324 is placed in connection with the end part of the telescope arranged electrode 325 to make sure the treatment takes places as close to the outlet as possible. The outer electrode 320 could be cylindrical, cone formed, square or any other form, e.g. copy of the telescope structure.

FIG. 33 shows where only one section 326 of the inner electrode is exposed. When only the first section of the inner electrode is exposed, the distance 328 between the two electrodes 326 and 327 is greater than the distance between the electrodes shown on FIG. 32.

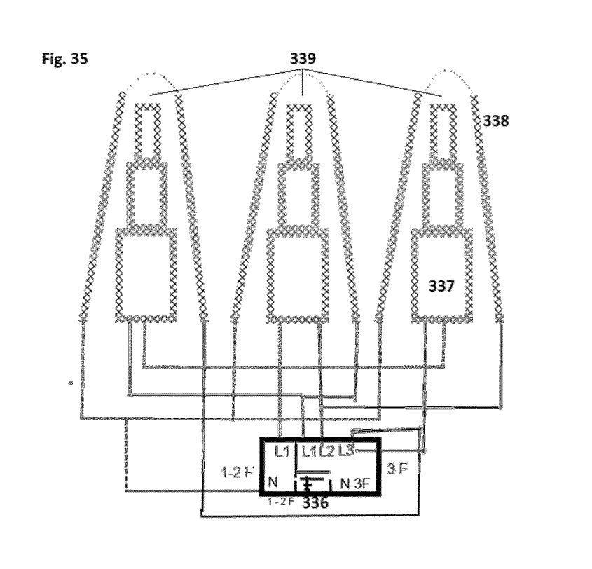

FIG. 34 show a cross view of the divide inner electrode 329 and the connector to the power supply 330 and the outer electrode 331 and in specific how the divided parts of the electrode that is arrange in a telescope like way connects to each other via shoulder 332 and a recess 333 which in this design works not only to keep the different parts of the electrode in place put to at the same time connect to each other such that power or current can pass from one part to the other. The connector that is attached to the one end of the telescope arranged electrode which is in a fixed position 334 also connects to the power via 330. 335 shows the units backside.

FIG. 35 illustrates the electrical wiring for a device. The device is able to use 1, 2 or 3 phases with simple wiring in the connection box.

For 1 Phase Connection 1f

An extension wire with phase, neutral and ground is used. The phase will be connected to L1 for 1-2f, neutral will be connected to both N1-2f and N3f, ground will be connected to the common ground/earth 336.

This way e.g. all inner electrodes 337 will be connected to the phase and e.g. all outer electrodes 338 will be connected to neutral thus creating an electrical potential between the two electrodes. Ground is connected to the outlet and inlet for current leakages. N3f is neutral for e.g. automated control.

For 2 Phases Connection 2f

An extension wire with 2 phases, neutral and ground is used. The 2 phases will be connected to L1 for 1-2f and N1-2f, neutral will be connected to N3f and ground connected to the common ground/earth 336.

This way the inner and outer electrodes will be connected to two different phases thus creating an electrical potential between the two electrodes. Ground is connected to the outlet and inlet for current leakages. N3f is neutral for e.g. automated control.

For 3 Phases Connection 3f

An extension wire with 3 phases, neutral and ground is used. The 3 phases will be connected to L1, L2 and L3 for 3f, neutral will be connected to N3f and ground connected to the common ground/earth 336.

This way the 3 phases will be evenly divided on the 3 chambers 339. The inner 337 and outer 338 electrodes will be connected to two different phases thus creating an electrical potential between the two electrodes. Ground is connected to the outlet and inlet for current leakages. N3f is neutral for e.g. automated control.

### EXAMPLES/TEST RESULTS

The below examples and test results illustrate different possibilities of the use of an embodiment of a device according to the invention. It should be understood that the test results are given by way of illustration only. Further, the official tests which have been carried out are in Danish language. To use the original documents, an English translation of the words are provided below the original test transcripts.

The below table 1 includes test results from three different measurements 1, 2, and 3 in which the device has been used to heat a liquid in the form of tap water.

TABLE 1

| | Inlet temp. | Outlet temp. | | Consumed | Delivered | |
|---|---|---|---|---|---|---|
| | [degrees] | [degrees] | Flow [l/h] | power [kW] | power [kW] | COP* |
| 1 | 9.3 | 14.7 | 1782 | 10.97 | 11.18 | 1.019 |
| 2 | 9.1 | 19.5 | 1171 | 13.93 | 14.07 | 1.010 |
| 3 | 8.6 | 14.7 | 1785 | 12.44 | 12.72 | 1.023 |

*Coefficient of performance

Table 2 and 3 illustrate removal of Legionella bacteria. The value of Table 2 is from regular tap water containing too much Legionella. Table 3 contains the Legionella value for tap water from the same site. The water has been treated by use of an embodiment of the device according to the invention. As shown in the tables, the number of Legionella bacteria is decreased from 10000 per litre to below 10 per litre, while the temperature of the water is increased from 46 degrees to 62 degrees.

TABLE 2

| Prøvenr.: C0371701 | | | | | |
|---|---|---|---|---|---|
| Prøve ID: | | | Detekt. | | Um |
| Prøvemærke: Køkkenhane | | | grænse | Metoder | (%) |
| Legionella | 10000 | antal/l | 10 | DS 3029 | |

| | | | |
|---|---|---|---|
| Oplysninger fra | 46.0 | gr. C. | [symbol] |
| rekvirenten: | | | |
| Vandtemperatur | | | |

[symbol]

indicates data missing or illegible when filed

Translation from Danish to English:

Prøvenr.:=Samle No, Prøve ID=Sample ID, Prøvemærke=testseal, Køkkenhane=kitchen faucet, Detekt. Grænse=detcection limit, Metoder=method, antal/L=parts/L, Oplysninger fra rekvirenten, information from the requestor, Vandtemperatur=water temperature, gr. C=degrees celsius.

TABLE 3

| Prøvenr.: 10629368 | | | | | |
|---|---|---|---|---|---|
| Prøve ID: | | | Detekt. | | Um |
| Prøvemærke: | | | grænse | Metoder | (%) |
| Legionella | <10 | antal/l | 10 | DS 3029 | |

| | | | |
|---|---|---|---|
| Oplysninger fra | 62.0 | gr. C. | [symbol] |
| rekvirenten: | | | |
| Vandtemperatur | | | |

[symbol]

indicates data missing or illegible when filed

Translation from Danish to English:

Prøvenr.:=Samle No, Prøve ID=Sample ID, Prøvemærke=testseal, Køkkenhane=kitchen faucet, Detekt. Grænse=detcection limit, Metoder=method, antal/L=parts/L, Oplysninger fra rekvirenten, information from the requestor, Vandtemperatur=water temperature, gr. C=degrees celsius.

Table 4 and 5 illustrate removal of bacteria (Kimtal=total bacterial count). The values of Table 4 are from lake water containing too many bacteria. Table 5 contains the values for lake water from the same site. The lake water has been treated by use of an embodiment of the device according to the invention. As shown in the tables, the total bacterial count (Kimtal) is decreased from above 3000 to 400 at 22 degrees, and from 3000 to 47 at 37 degrees.

TABLE 4

| | | | |
|---|---|---|---|
| Prøvenr.: C7072901 | | | |
| Prøve ID: | Detekt. | | Um |
| Prøvenmærke: | grænse | Metoder | (%) |

| | | | | |
|---|---|---|---|---|
| Prøvens farve | gul | | | *VISUEL |
| Prøvens klarhed | sv. uklar | | | *VISUEL |
| Prøvens lugt | ubehag. | | | *ORGANOLEP |
| Coliforme bakterier 37° C. | 11 | M [symbol] N/100 ml | 1 | Colilert [symbol] |
| E. coli | <1 | M [symbol] N/100 ml | 1 | Colilert [symbol] |
| Kimtal ved 22° C., GEA | >3000 | CFU/ml | 1 | DS/I 6222:2000 |
| Kimtal ved 37° C., GEA | 3000 | CFU/ml | 1 | DS/I 6222:2000 |

[symbol]

indicates data missing or illegible when filed

Translation from Danish to English:

Prøvenr.:=Samle No, Prøve ID=Sample ID, Prøvemærke=test seal, Køkkenhane=kitchen faucet, Detekt. Grænse=detection limit, Metoder=method, Prøvens farve=Color of the sample, Prøvens klarhed=Sample clarity, Prøvens lugt=the smell of the sample, Coliforme bakterier=Coliform bacteria, Kimtal ved 22 C=total bacteria number at 22 degrees Celsius, Kimtal ved 37 C=total bacteria number at 37 degrees Celsius, gul=yellow, sv. Uklar=very unclear, ubehag=discomfort.

TABLE 5

| | | | |
|---|---|---|---|
| Prøvenr.: C7072701 | | | |
| Prøve ID: | Detekt. | | Um |
| Prøvemærke: | grænse | Metoder | (%) |

| | | | | |
|---|---|---|---|---|
| Prøvens farve | gul | | | *VISUEL |
| Prøvens klarhed | sv. uklar | | | *VISUEL |
| Prøvens lugt | ubehag. | | | *ORGANOLEP |
| Coliforme bakterier 37° C. | <1 | MPN/100 ml | 1 | Colilert [symbol] |
| E. coli | <1 | MPN/100 ml | 1 | Colilert [symbol] |
| Kimtal ved 22° C., GEA | 400 | CFU/ml | 1 | DS/I 6222:2000 |
| Kimtal ved 37° C., GEA | 47 | CFU/ml | 1 | DS/I 6222:2000 |

[symbol]

indicates data missing or illegible when filed

Translation from Danish to English:

Prøvenr.:=Samle No, Prøve ID=Sample ID, Prøvemærke=test seal, Køkkenhane=kitchen faucet, Detekt. Grænse=detcection limit, Metoder=method, Prøvens farve=Color of the sample, Prøvens klarhed=Sample clarity, Prøvens lugt=the smell of the sample, Coliforme bakterier=Coliform bacteria, Kimtal ved 22 C=total bacteria number at 22 degrees Celsius, Kimtal ved 37 C=total bacteria number at 37 degrees Celsius, gul=yellow, sv. Uklar=very unclear, ubehag=discomfort.

Table 6, 7 and 8 illustrate removal of bacteria (Kimtal=total bacterial count), substantially without affecting the pH value of the liquid. The values of Table 6 are from regular tap water containing too many bacteria. Table 7 and 8 contain the values for tap water from the same site. The tap water has been treated by use of an embodiment of the device according to the invention. The water of Table 7 has been heated approximately 9 degrees, while the water of Table 8 has been heated approximately 20 degrees. The pH value of the water is changed from 7.7 to 7.6 and 7.5, respectively.

TABLE 6

| Prøvenr.: 23268226 | | | | | |
|---|---|---|---|---|---|
| Prøve ID: | | | Detekt. | | Um |
| Prøvenmærke: | | | grænse | Metdoer | (%) |
| Coliforme bakterier 37° C. | 1 | MPN/100 ml | 1 | Colilert [symbol] | |
| E. coli | <1 | MPN/100 ml | 1 | Colilert [symbol] | |
| Kimtal ved 22° C., GEA | >3000 | CFU/ml | 1 | DS/I 6222:2000 | |
| Kimtal ved 37° C., GEA | >3000 | CFU/ml | 1 | DS/I 6222:2000 | |
| Hårdhed, total | 14.1 | H grader | 0.5 | SM3120-ICP | 30 |
| Calcium (Ca) | 89 | mg/l | 0.50 | SM3120-ICP | 30 |
| Magnesium (Mg) | 7.2 | mg/l | 0.10 | SM3120-ICP | 30 |
| Kalium (K) | 2.1 | mg/l | 0.20 | SM3120-ICP | 30 |
| Natrium (Na) | 17 | mg/l | 0.10 | SM3120-ICP | 30 |
| Jern (Fe) | 0.055 | mg/l | 0.010 | SM3120-ICP | 30 |
| Mangan (Mn) | <0.005 | mg/l | 0.005 | SM3120-ICP | 30 |
| Ammonium | 0.008 | mg/l | 0.006 | SM 17.udg. 4500 | 10 |
| Nitrit | <0.005 | mg/l | 0.005 | SM 17.udg. 4500 | 10 |
| Nitrat | 2.0 | mg/l | 0.50 | SM 17.udg. 4500 | 10 |
| Total-P | 0.031 | mg/l | 0.005 | DS/EN I 6878 aut | 10 |
| Chlorid | 32 | mg/l | 1.00 | SN 17.udg. 4500 | 10 |
| Fluorid | 0.19 | mg/l | 1.00 | SN 17.udg. 4500 | 10 |
| Sulfat | 50 | mg/l | 0.050 | SN 17.udg. 4500 | 10 |
| Hydrogencarbonat | 255 | mg/l | 0.50 | DS/EN I 9963 | 10 |
| Aggressiv kuldioxid | <5 | mg/l | 3.0 | DS 236:1977 | 20 |
| Turbiditet | 0.34 | [symbol] TU | 5 | DS/EN I 7027 | 20 |
| Farvetal, Pt | 3.1 | mgPt/l | 0.10 | DS/EN I 6271-2 | 10 |
| Inddampningsrest | 370 | mg/l | 1.0 | DS 204:1980 | 12 |
| NVOC, ikke flygt.org.carbon | 1.1 | mg/l | 0.10 | DS/En 1484 | 12 |

[symbol]

indicates data missing or illegible when filed

Translation from Danish to English:

Prøvenr.:=Samle No, Prøve ID=Sample ID, Prøvemærke=test seal, Køkkenhane=kitchen faucet, Detekt. Grænse=detcection limit, Metoder=method, Coliforme bakterier 37 C=Coliforme bacteria at 37 degrees Celsius, Kimtal ved 22 C, total bacteria number at 22 degrees Celsius, Kimtal ved 37 C, total bacteria number at 37 degrees Celsius, hårdhed=hardness, Calcium=kalium=potassium, natrium=Sodium, jern=iron, mangan=manganese, nitrit=nitrite, nitrat=nitrate, chlorid=chloride, fluorid=floride, sulfat=sulphate, aggressive kuldioxid=aggressive carbon dioxide, turbiditet=turbidity, farve tal=color figures, inddampningsrets=evaporation residue, NVOC—ikke flygt. Org. carbon=NVOC—Nonvolatile organic carbon.

Oplysninger fra prøvetageren

| Prøvens farve | farveløs | | | *VISUEL | |
|---|---|---|---|---|---|
| Prøens klarhed | klar | | | *VISUEL | |
| Prøvens lugt | ingen | | | *ORGANOLEP | |
| Vandtemperatur | 16.9 | gr. C. | | DS2250 | |
| pH | 7.7 | pH | | DS 267:1976 | 10 |
| Ledningsevne | 57 | mS/m | 0.1 | DS/EN 278 [symbol] | 6 |
| Iltindhold | 10.7 | mg/l | 0.1 | DS/EN 25814 | 6 |

[symbol]

indicates data missing or illegible when filed

Translation from Danish to English:

Oplysninger fra prøvetageren=Information from the test receiver, Prøvens farve=color of the sample, Prøvens klarhed=Clarity of the sample, Prøvens lugt=the smell of the sample, Vandtemperatur=water temperature, Ledningsevne=conductivity, iltindhold=oxygen content, farveløs=color less, sv. Uklar=very unclear, ingen=none, gr. C=degrees Celsius

TABLE 7

| Prøvenr.: 23268225 | | | | | |
|---|---|---|---|---|---|
| Prøve ID: | | | Detekt. | | Um |
| Prøvemærke: | | | grænse | Metoder | (%) |
| Coliforme bakterier 37° C. | <1 | MPN/100 ml | 1 | Colilert [symbol] | |
| E. coli | <1 | MPN/100 ml | 1 | Colilert [symbol] | |
| Kintal ved 22° C., GEA | 4 | CFU/ml | 1 | DS/I 6222:2000 | |
| Kintal ved 37° C., GEA | <1 | CFU/ml | 1 | DS/I 6222:2000 | |
| Hårdhed, total | 13.9 | H grader | 0.5 | SM3120-ICP | 30 |
| Calcium (Ca) | 87 | mg/l | 0.50 | SM3120-ICP | 30 |
| Magnsium (Mg) | 7.2 | mg/l | 0.10 | SM3120-ICP | 30 |
| Kalium (K) | 2.1 | mg/l | 0.20 | SM3120-ICP | 30 |
| Natrium (Na) | 17 | mg/l | 0.10 | SM3120-ICP | 30 |
| Jern (Fe) | 1.9 | mg/l | 0.010 | SM3120-ICP | 30 |
| Mangan (Mn) | 0.035 | mg/l | 0.005 | SM3120-ICP | 30 |
| Ammonium | 0.009 | mg/l | 0.006 | SM 17.udg. 4500 | 10 |
| Nitrit | <0.005 | mg/l | 0.005 | SM 17.udg. 4500 | 10 |
| Nitrat | 0.74 | mg/l | 0.50 | SM 17.udg. 4500 | 10 |
| Total-P | 0.031 | mg/l | 0.005 | DS/EN I 6878 aut | 10 |
| Chlorid | 31 | mg/l | 1.00 | SM 17.udg. 4500 | 10 |
| Fluorid | 0.23 | mg/l | 0.050 | SM 17.udg. 4500 | 10 |
| Sulfat | 49 | mg/l | 0.50 | SM 17.udg. 4500 | 10 |
| Hydrogencarbonat | 256 | mg/l | 3.0 | DS/EN I 9963 | 10 |
| Aggressiv kuldioxid | <5 | mg/l | 5 | DS 236:1977 | 20 |
| Tubiditet | 2.2 | FTU | 0.10 | DS/EN I 7027 | 20 |
| Farvetal, Pt | 10 | mgPt/l | 1.0 | DS/EN I 6271-2 | 10 |
| Inddampningsrest | 370 | mg/l | 10 | DS 204:1980 | 12 |
| NVOC, ikke flygt.org.carbon | 1.4 | mg/l | 0.10 | DS/EN 1484 | 12 |

[symbol]

indicates data missing or illegible when filed

Translation from Danish to English:

Prøvenr.:=Samle No, Prøve ID=Sample ID, Prøvemærke=test seal, Køkkenhane=kitchen faucet, Detekt. Grænse=detcection limit, Metoder=method, Coliforme bakterier 37 C=Coliforme bacteria at 37 degrees Celsius, Kimtal ved 22 C, total bacteria number at 22 degrees Celsius, Kimtal ved 37 C, total bacteria number at 37 degrees Celsius, hågrdhed=hardness, Calcium=kalium=potassium, natrium=Sodium, jern=iron, mangan=manganese, nitrit=nitrite, nitrat=nitrate, chlorid=chloride, fluorid=floride, sulfat=sulphate, aggressive kuldioxid=aggressive carbon dioxide, turbiditet=turbidity, farve tal=color figures, inddampningsrets=evaporation residue, NVOC—ikke flygt. Org. carbon=NVOC—Nonvolatile organic carbon.

Oplysninger fra prøvetageren:

| Prøvens farve | farveløs | | | *VISUEL | |
|---|---|---|---|---|---|
| Prøvens klarhed | sk. uklar | | | *VISUEL | |
| Prøvens lught | ingen | | | *ORGANOLEP | |
| Vandtemperatur | 29.0 | gr. C. | | DS2250 | |
| pH | 7.6 | pH | | DS 287:1978 | 10 |
| Ledningsevne | 57 | mS/m | 0.1 | DS/EN 27888 | 6 |
| Iltindhold | 8.4 | mg/l | 0.1 | DS/EN 25814 | 6 |

Translation from Danish to English:

Oplysninger fra prøvetageren=Information from the test receiver, Prøvens farve=color of the sample, Prøvens klarhed=Clarity of the sample, Prøvens lugt=the smell of the sample, Vandtemperatur=water temperature, Ledningsevne=conductivity, iltindhold=oxygen content, farveløs=color less, sv. Uklar=very unclear, ingen=none, gr. C=degrees Celsius

TABLE 8

| Prøvenr.: | | | | | |
|---|---|---|---|---|---|
| Prøve ID: | | | Detekt. | | Um |
| Prøvemærke: | | | grænse | Metoder | (%) |
| Coliforme bakterier 37° C. | <1 | MPN/100 ml | 1 | Colilert [symbol] | |
| E. coli | <1 | MPN/100 ml | 1 | Colilert [symbol] | |
| Kimtal ved 22° C., GEA | <1 | CFU/ml | 1 | DS/I 6222:2000 | |
| Kimtal ved 37° C., GEA | <1 | CFU/ml | 1 | DS/I 6222:2000 | |
| Hådhed, total | 13.7 | H grader | 0.5 | SM3120-ICP | 30 |
| Calcium (Ca) | 86 | mg/l | 0.50 | SM3120-ICP | 30 |
| Magnesium (Mg) | 7.3 | mg/l | 0.10 | SM3120-ICP | 30 |
| Kalium (K) | 2.1 | mg/l | 0.20 | SM3120-ICP | 30 |
| Natrium (Na) | 17 | mg/l | 0.10 | SM3120-ICP | 30 |
| Jern (Fe) | 2.7 | mg/l | 0.010 | SM3120-ICP | 30 |
| Mangan (Mn) | 0.046 | mg/l | 0.005 | SM3120-ICP | 30 |
| Ammonium | <0.006 | mg/l | 0.006 | SM 17.udg. 4500 | 10 |
| Nitrit | <0.005 | mg/l | 0.005 | SM 17.udg. 4500 | 10 |
| Nitrat | 0.92 | mg/l | 0.50 | SM 17.udg. 4500 | 10 |
| Total-P | 0.032 | mg/l | 0.005 | DS/EN I 6878 aut | 10 |
| Chlorid | 32 | mg/l | 1.00 | SM 17.udg. 4500 | 10 |
| Fluorid | 0.23 | mg/l | 0.050 | SM 17.udg. 4500 | 10 |
| Sulfat | 47 | mg/l | 0.50 | SM 17.udg. 4500 | 10 |
| Hydrogencarbonat | 256 | mg/l | 3.0 | DS/EN I 9963 | 10 |
| Aggressiv kuldioxid | <5 | mg/l | 5 | DS 236:1977 | 20 |
| Turbiditet | 7.6 | [symbol] TU | 0.30 | DS/EN I 7027 | 20 |
| Farvetal, Pt | 12 | mgPt/l | 1.0 | DS/EN I 6271-2 | 10 |
| InddampninsGest | 370 | mg/l | 10 | DS 204:1980 | 12 |
| NVOC, ikke flygt.orb.carbon | 1.3 | mg/l | 0.10 | DS/EN I484 | 12 |

[symbol]

indicates data missing or illegible when filed

Translation from Danish to English:

Prøvenr.:=Samle No, Prøve ID=Sample ID, Prøvemærke=testseal, Køkkenhane=kitchen faucet, Detekt. Grænse=detcection limit, Metoder=method, Coliforme bakterier 37 C=Coliforme bacteria at 37 degrees Celsius, Kimtal ved 22 C, total bacteria number at 22 degrees Celsius, Kimtal ved 37 C, total bacteria number at 37 degrees Celsius, hårdhed=hardness, Calcium=kalium=potassium, natrium=Sodium, jern=iron, mangan=manganese, nitrit=nitrite, nitrat=nitrate, chlorid=chloride, fluorid=floride, sulfat=sulphate, aggressive kuldioxid=aggressive carbon dioxide, turbiditet=turbidity, farve tal=color figures, inddampningsrets=evaporation residue, NVOC—ikke flygt. Org. carbon=NVOC—Nonvolatile organic carbon.

Oplysninger fra prøvetageren:

| Prøvens farve | farveløs | | | *VISUEL | |
|---|---|---|---|---|---|
| Prøvens klarhed | sv.uklar | | | *VISUEL | |
| Prøvens lugt | ingen | | | *ORGANOLEP | |
| Vandtemperatur | 37.0 | gr. C. | | DS2250 | |
| pH | 7.5 | pH | | DS 287:1978 | 10 |
| Ledningsevne | 57 | mS/m | 0.1 | DS/EN 27888 | 6 |
| Iltindhold | 7.7 | mg/l | 0.1 | DS/EN 25814 | 6 |

Translation from Danish to English:

Oplysninger fra prøvetageren=Information from the test receiver, Prøvens farve=color of the sample, Prøvens klarhed=Clarity of the sample, Prøvens lugt=the smell of the sample, Vandtemperatur=water temperature, Ledningsevne=conductivity, iltindhold=oxygen content, farveløs=color less, sv. Uklar=very unclear, ingen=none, gr. C=degrees Celsius.

Tabel 9 displays testresults from testing the treatment device on a pure culture of E. coli as well as on effluent wastewater, each resembling different complexity. The pure culture was freshly produced prior to the experiment and the wastewater was collected on day of trial from the secondary clarifier at a wastewater treatment plant. The treatment effect was assessed on the numbers of E. coli, coliform bacteria, Enterorocci and total aerobic bacteria. The prototype was installed in a laboratory at Danish Technology Institut where the inoculum was pumped through the device, which was adjusted to 3000 Watt—maximum current from a 1 fase 220 V outlet.

Tabel 10 shows that tests at 2950 watts had a bactericidal effect on all the bacteria cultivated from the wastewater. No coliforms, E. coli and Enterococci could be detected after treatmemt, corresponding to a log 2 and log 3 reduction, respectively.

Tabel 11 shows studies on H2 production with the device treating tap water. Unisense H2 microsenser—miniaturized Clark-type sensor calibrated at different temperatures. Data was logged in SensorTrace Basic. The results showed that heating tap water in the range from 18-55 degrees using the device significantly increased the H2 concentration in the water. Example an increase from 0 uM to 91,3 uM at 1400 Watt was measured.

1-67. (canceled)

68. An instant type liquid treating device comprising a housing with an inlet and an outlet connected by a passage for passing a liquid medium through the device, the device comprising at least one set of conductive faces one of which is formed by an outer electrode and one of which is formed by an inner electrode, the outer electrode forming a channel in which the inner electrode is received, the faces being connectable to an AC-power supply and being directly exposed to each other in the passage such that the liquid medium can be treated by way of its own electrical resistance between the faces, characterised in that at least one of the outer and the inner electrode comprises at least two individual elements axially movable relative to each other via a control which thereby facilitate relative movement of the faces.

69. A device according to claim 68, where the control facilitates both a change in an amount of confronting areas of the faces and spacing between the confronting faces.

70. A device according to claim 68, wherein one of the confronting faces comprises at least a surface portion which is non-parallel to at least a portion of the other face.

71. A device according to claim 68, wherein the faces are shifted in linear relationship.

72. A device according to claim 68, where the at least two individual elements of the outer or inner electrode are cylindrical elements.

73. A device according to claim 68, where at least one of the outer electrode and the inner electrode comprises a conical section.

74. A device according to claim 72, where both the outer electrode and the inner electrode are cylindrical and coaxial.

75. A device according to claim 74, where relative movement of the faces at least partly is provided by relative movement of the at least two individual cylindrical elements constituting the outer electrode or the inner electrode.

76. A device according to claim 68 where at least one of the inner electrode and the outer electrode is a telescope-like electrode where the at least two individual elements are arranged in a telescopic layout relative to each other.

77. A device according to claim 76, where both electrodes are conical or are telescope-like whereby they can become conical when brought into an expanded state, the conical electrodes having a narrow end and an opposite wide end, the narrow ends of both electrodes being in the same direction.

78. A device according to claim 68, comprising at least one extension-tube of a non-conductive material extending from at least one of the inlet and the outlet, the extension tube terminating in a coupler of a conductive material, the coupler being electrically connectable to a consumer of electricity or to ground of a power supply or to zero of a power supply.

79. A device according to claim 68, comprising an AC-power supply providing an AC signal and connected to the inner and outer electrodes to provide a potential difference between the faces, the potential difference varying with the AC signal.

80. A device according to claim 79, where the AC-power supply further comprises an electronic circuit facilitating phase angle control or pulse generation.

81. A device according to claim 68, further comprising a sensor for sensing a parameter significant for the liquid medium, and means for adjusting at least one of the power supply and the relative movement of the faces based on the sensed parameter.

82. A device according to claim 68, where the faces are located relative to the passage such that an entire amount of the liquid medium flowing between the inlet and the outlet passes between the faces.

83. A device according to claim 68, comprising a shunt loop facilitating return of at least a portion of liquid medium which exits the outlet back to the inlet.

84. A device according to claim 68, comprising several passages and corresponding sets of separate conductive faces, the passages being arranged in parallel between the inlet and outlet.

85. A device according to claim 68, further comprising a controlled valve located to control the flow of the liquid medium through the passage or wherein the controlled valve is controlled based on a control parameter which is indicative of:
a desired flow of the liquid medium through the passage; or
a desired temperature of the liquid medium; or
a desired reduction of a microbial count in the liquid medium; or
a desired increase in content of hydrogen in the liquid medium; or
a desired reduction in the limescale developing effect of the liquid medium.

86. A method for treating a liquid medium where the liquid medium is exposed to an electrical field by use of the device according to claim 68, wherein the liquid medium is exposed to the electrical field until a reduction of a microbial count can be detected, or the liquid medium is exposed to the electrical field until the content of hydrogen in the liquid medium is increased, or the liquid medium is exposed to the electrical field until the limescale developing effect of the liquid medium is decrease relative to the limescale developing effect of the liquid medium before it is exposed to the electrical field.

87. A method according to claim 86, where the liquid medium is a body fluid in vitro, a content of a beverage, or process water for cleaning purpose or for watering of plants.
